(19) United States
(12) Patent Application Publication
(10) Pub. No.: US 2024/0307400 A1
(43) Pub. Date: Sep. 19, 2024

(54) COMPOSITIONS AND METHODS FOR THE PREVENTION AND TREATMENT OF HEARING LOSS

(71) Applicant: TING THERAPEUTICS LLC, Carlsbad, CA (US)

(72) Inventors: Jian ZUO, Carlsbad, CA (US); Tal TEITZ, Omaha, NE (US)

(21) Appl. No.: 18/676,439

(22) Filed: May 28, 2024

Related U.S. Application Data
(63) Continuation-in-part of application No. 17/736,330, filed on May 4, 2022.
Application No. 17/736,330 is a division of application No. 17/580,755, filed on Jan. 21, 2022, now Pat. No. 11,433,073.
Application No. 17/580,755 is a continuation of application No. PCT/US2020/063595, filed on Dec. 7, 2020.
(60) Provisional application No. 62/947,059, filed on Dec. 12, 2019.

(51) Int. Cl. A61K 31/519; A61K 31/437; A61K 31/4439; A61K 31/4709; A61K 31/506; A61P 27/16

(52) U.S. Cl. CPC A61K 31/519; A61K 31/437; A61K 31/4439; A61K 31/4709; A61K 31/506; A61P 27/16

(57) ABSTRACT

This disclosure provides for methods, kits and pharmaceutical compositions comprising an inhibitor of EGFR signaling for the treatment of hearing loss. In particular, one such EGFR inhibitor, Dabrafenib, is a therapeutic candidate for treating cisplatin-induced and/or noise-induced hearing loss.

### CROSS-REFERENCES TO RELATED APPLICATIONS

This application is a Continuation-in-Part of U.S. application Ser. No. 17/736,330 filed May 4, 2022, which was a Division of U.S. application Ser. No. 17/580,755 filed Jan. 21, 2022, which was a Continuation of PCT/US2020/063595 filed Dec. 7, 2020, which claim priority to U.S. Provisional Application No. 62/947,059 filed Dec. 12, 2019, each of which are incorporated herein by reference.

### STATEMENT REGARDING FEDERALLY SPONSORED RESEARCH OR DEVELOPMENT

This invention was made with government support under Grant Numbers DC006471 DC015010, DC015444, DC013879, DC013232, DC018850 and CA021765 awarded by the National Institutes of Health and Grant Numbers N00014-09-V-1014, N00014-12-V-0191, N00014-12-V-0775, awarded by the National Institute of Health and N00014-16-V-2315 awarded by the Office of Naval Research. The government has certain rights in the invention.

### FIELD

The invention relates to compositions and methods for the treatment or prevention of hearing loss by administering to an animal in need thereof an inhibitor of epidermal growth factor receptor (EGFR) signaling.

### BACKGROUND

The ear is a complex organ composed of a labyrinth of structures responsible for hearing and balance. Perception of both hearing and balance lies in the ability of inner ear structures to transform mechanical stimuli to impulses recognized by the brain. The sensory receptors responsible for hearing are located in the cochlea, a spiral-shaped canal filled with fluid. Within the cochlea is the organ of Corti, which is lined with columnar sensory hair cells bridging the basilar membrane and the tectorial membrane. As sound waves pass through the organ of Corti, the basilar membrane vibrates causing the hair cells to bend back and forth. The movement depolarizes the hair cell, leading to release of neurotransmitters to the auditory nerve, which carries the impulse to the brain.

Cisplatin is a highly effective and commonly used chemotherapy agent for the treatment of a variety of cancers, but 40-60% of patients treated with cisplatin have irreversible hearing loss. Cisplatin negatively affects high frequency hearing more than lower frequencies primarily due to death of outer hair cells (OHCs) in the cochlear basal turn. Hair cells are the most common cochlear cell type to be affected by cisplatin but cells of the stria vascularis, spiral ganglion neurons, and supporting cells have also been reported to suffer deleterious effects. Cisplatin-induced hearing loss negatively impacts an individual's quality of life, leading to depression and social isolation, and impeding the development of language skills in young children treated with cisplatin. There is a clinical need to develop drugs that can protect from this highly common side effect of cisplatin treatment.

Currently, there is only one Food and Drug Administration (FDA)-approved drug for the treatment of cisplatin ototoxicity which has limited application. Sodium thiosulfate (STS) was approved by the FDA to reduce the risk of cisplatin-induced ototoxicity in pediatric patients one month or older with localized, non-metastatic solid tumors, and represents a significant advancement in the field of hearing loss prevention. STS is administered to patients six hours after cisplatin treatment due to concerns over its interference with cisplatin's tumor killing efficacy even though no conclusive data demonstrates direct interference and no difference in hearing outcomes is observed with the delay in treatment. Recently, the antioxidant N-acetylcysteine (NAC) was shown to be otoprotective in a phase-1 clinical trial in children and adolescents diagnosed with localized, nonmetastatic, cisplatin treated tumors. No severe adverse events occurred following N-acetylcysteine treatment which makes it a promising compound for the treatment of cisplatin-induced hearing loss.

### BRIEF SUMMARY

The invention provides a compositions and method to treat hearing loss by orally administering to subject in need thereof a pharmaceutical composition made of: a therapeutically effective amount of dabrafenib to prevent hearing loss due to cisplatin treatment, administered for one or a plurality of dose cycles to the subject. In one embodiment, Dabrafenib is administered twice a day to the subject. In some aspects, the subject is first administered an amount of cisplatin at a dose of about 30 mg/kg.

The combinations provided herein for the treatment of hearing loss exhibit excellent properties including a low effective dose, a good toxicity profile, a therapeutic index of at least 25 in the multi-dose cisplatin regimen, protecting both female and male subjects, reducing hearing loss in two different strains of mice (FVB/NJ and CBA/CaJ), and protection from weight loss and kidney damage that typically occurs during cisplatin chemotherapy, and persistence of hearing treatment for at least four months after cisplatin treatments.

In some aspects, this disclosure provides for a method to prevent cisplatin-induced hearing loss including the steps of: orally administering to subject in need thereof a pharmaceutical composition made of: a therapeutically effective amount of dabrafenib to prevent hearing loss due to cisplatin treatment, wherein the therapeutically effective amount of dabrafenib is administered to the subject in a first cycle. In some aspects, the therapeutically effective amount of dabrafenib administered to the subject in the first cycle can be in a range from about 0.6 mg/kg to about 60 mg/kg in mice or a human equivalent dose. In some aspects, the therapeutically effective amount of dabrafenib in the first cycle can be about 12 mg/kg in mice or a human equivalent dose. In some aspects, the treatment amount of cisplatin can be about 30 mg/kg in mice or a human equivalent dose. In some aspects, dabrafenib is administered forty-five minutes prior to the cisplatin treatment. In some aspects, dabrafenib can be co-administered with the cisplatin treatment.

In some aspects, the method can further include the step of orally administering to subject in need thereof a pharmaceutical composition is made of: a sufficient amount of dabrafenib to prevent hearing loss due to cisplatin treatment, wherein the sufficient amount of dabrafenib is about 12 mg/kg in mice or a human equivalent dose for a second cycle after a rest period.

In some aspects, the method can further include the step of orally administering to subject in need thereof a pharmaceutical composition is made of: a sufficient amount of dabrafenib to prevent hearing loss due to cisplatin treatment, wherein the sufficient amount of dabrafenib is about 12 mg/kg in mice or a human equivalent dose for a third cycle after a rest period.

In some aspects, this disclosure provides for a method to prevent cisplatin-induced hearing loss comprising: orally administering to subject in need thereof a pharmaceutical composition is made of: a sufficient amount of dabrafenib to prevent hearing loss due to cisplatin treatment, wherein the sufficient amount of dabrafenib can be in a range from 0.6 mg/kg to 60 mg/kg in mice or a human equivalent dose. In some aspects, the treatment amount of cisplatin can be about 30 mg/kg in mice or a human equivalent dose. In some aspects, dabrafenib can be administered forty-five minutes prior to the cisplatin treatment. In some aspects, dabrafenib can be co-administered with the cisplatin treatment.

In some aspects, the method can further include the step of orally administering to subject in need thereof a pharmaceutical composition is made of: a sufficient amount of dabrafenib to prevent hearing loss due to cisplatin treatment, wherein the sufficient amount of dabrafenib is about 3 mg/kg in mice or a human equivalent dose for a second cycle after a rest period.

In some aspects, the method can further include the step of orally administering to subject in need thereof a pharmaceutical composition is made of: a sufficient amount of dabrafenib to prevent hearing loss due to cisplatin treatment, wherein the sufficient amount of dabrafenib is about 3 mg/kg in mice or a human equivalent dose for a third cycle after a rest period.

In some aspects, the method can further include the step of administering to the subject a therapeutically effective amount of AZD5438. The therapeutically effective amount of AZD5438 can range from about 5 mg/kg to about 85 mg/kg, preferably about 35 mg/kg.

In some aspects, this disclosure provides for a method to prevent cisplatin-induced hearing loss comprising: orally administering to subject in need thereof a pharmaceutical composition is made of: a sufficient amount of dabrafenib to prevent hearing loss due to cisplatin treatment, wherein the sufficient amount of Dabrafenib can be about 0.6 mg/kg in mice or a human equivalent dose. In some aspects, the treatment amount of cisplatin can be about 30 mg/kg in mice or a human equivalent dose. In some aspects, dabrafenib can be administered forty-five minutes prior to the cisplatin treatment. In some aspects, dabrafenib can be administered simultaneously with the cisplatin treatment.

In some aspects, the method can further include the step of orally administering to subject in need thereof a pharmaceutical composition is made of: a sufficient amount of dabrafenib to prevent hearing loss due to cisplatin treatment, wherein the sufficient amount of dabrafenib can be about 0.6 mg/kg in mice or a human equivalent dose for a second cycle after a rest period.

In some aspects, the method can further include the step of orally administering to subject in need thereof a pharmaceutical composition is made of: a sufficient amount of dabrafenib to prevent hearing loss due to cisplatin treatment, wherein the sufficient amount of dabrafenib can be about 0.6 mg/kg in mice or a human equivalent dose for a third cycle after a rest period.

In some aspects, this disclosure provides for a method to maintain subject weight during cisplatin treatment comprising: co-administrating a sufficient amount of cisplatin for the desired treatment and a sufficient amount of a B-raf inhibitor to prevent weight loss during cisplatin treatment. In some aspects, minimal kidney damage is observed during cisplatin treatment by the co-administration of a sufficient amount of a B-raf inhibitor during said cisplatin treatment.

In some aspects, this disclosure provides for a pharmaceutical composition in a unit dose form comprising cisplatin and an effective amount of a B-raf inhibitor to treat hearing loss caused by the co-administration of cisplatin to a subject in need of treatment thereof wherein the B-raf inhibitor is selected from the group consisting of: Dabrafenib, Vemurafenib, PLX-4720 and RAF-265. In some aspects, the pharmaceutical composition can further include a sufficient amount of Trametinib to treat hearing loss due to cisplatin treatment. The sufficient amount of Trametinib can be about 2 mg/kg in mice or an equivalent dosage for a human twice daily. The pharmaceutical composition can further comprising a therapeutically effective amount of AZD5438.

The inventive subject matter further includes a pharmaceutical composition in a unit dose form made of cisplatin and an effective amount of a B-raf inhibitor to protect against hearing loss caused by the co-administration of cisplatin to a subject in need of treatment thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows that the EGFR inhibitor MUBRITINIB (whose structure is shown) protects against cisplatin-induced hair cell loss in mouse cochlear explants with IC₅₀ of 2.5 nM and LD₅₀ of >500 nM (Therapeutic Index of >200). Number of explants: 1-4 at each dose; FVB mouse cochlear explants were treated with 150 μM cisplatin and cochlear middle turns were analyzed; curve fitting with R² of 0.86. Note that IC₅₀ values of MUBRITINIB were consistent in all assays (HEI-OC1 cells and explants) demonstrating its specificity and potency.

FIG. 2 shows that the EGFR inhibitor Pelitinib (whose structure is shown) protects against cisplatin-induced hair cell loss. Pelitinib is an irreversible inhibitor of EGFR that exhibits protective effects against cisplatin-induced Caspase-3/7 activity in HEI-OC1 cells with IC₅₀ of 0.6 μM (cisplatin-Caspase-Glo 3/7) and LD₅₀ of >40 μM (CELLTITER-GLO).

FIG. 3 shows dose-response of dabrafenib (Dab) in mouse cochlear explants treated with or without cisplatin. Dab alone or Dab added 1 h before cisplatin (150 μM) to P3 FVB cochlear explants for 24 h. Number of explants for each dose of Dab was shown. ***: P<0.001; **: P<0.01; *: P<0.05 (Student's T-test) comparing to Cis alone.

FIG. 4A shows signaling cascade of RTKs (receptor tyrosine kinases), RAS, RAF, MEK, and ERK in cisplatin-induced hair cell death and current small molecule inhibitors for RAF, MEK and ERK in otoprotection.

FIG. 4B shows the activity of B-Raf inhibitor Vemurafenib to protect in the cochlear explant culture assay against cisplatin-induced hair cell death. Dose-responses of the compounds in P3 FVB mouse cochlear explants treated with or without cisplatin were shown. See FIG. 3 for meaning of other labels.

FIG. 4C shows the activity of B-Raf inhibitor Trametinib.

FIG. 4D shows the activity of B-Raf inhibitor PLX-4720.

FIG. 4E shows the activity of B-Raf inhibitor RAF-265.

FIG. 5A shows Dabrafenib mitigates cisplatin activated B-Raf signaling cascade in HEI-OC1 cells. Western blots show representative changes of each signaling molecule upon cisplatin and Dab treatment at specific time.

FIG. 5B shows Dabrafenib mitigates cisplatin activated B-Raf signaling cascade in HEI-OC1 cells. Western blots show representative changes of each signaling molecule upon cisplatin and Dab treatment at specific doses (5B)

FIG. 6A shows schedule of administration of dabrafenib (100 mg/kg) and cisplatin (30 mg/kg) to adult FVB mice (males and females).

FIG. 6B shows reduced ABR threshold shifts of 11.8-15.0 dB in average were recorded on day 21 after first day of cisplatin (30 mg/kg) and dabrafenib (100 mg/kg) co-treatment, mean±SEM, *, P<0.05, compared to cisplatin alone by two-way ANOVA followed by a Bonferroni comparison.

FIG. 7A shows schedule of administration of dabrafenib (100 mg/kg) and noise exposure to adult FVB mice (males and females).

FIG. 7B shows reduced ABR threshold shifts of 18.1-21.9 dB in average were recorded on day 14 after first day of dabrafenib (100 mg/kg) and noise exposure, mean±SEM, **, P<0.01, ***, P<0.001, compared to carrier by two-way ANOVA followed by a Bonferroni comparison.

FIG. 8A shows schedule of administration of dabrafenib (60 mg/kg×2 daily) and noise exposure to adult FVB mice (males and females).

FIG. 8B shows reduced ABR threshold shifts of 13.5-21.2 dB on average were recorded on day 14 after first day of dabrafenib (60 mg/kg×2 daily) and noise exposure, mean±SEM, **, P<0.01, ***, P<0.001, compared to carrier by two-way ANOVA followed by a Bonferroni comparison.

FIG. 9 shows Testing a B-Raf/MEK1/2 inhibitor combination in mouse cochlear explant cultures. Compounds alone or combination of the compounds were added 1 h before cisplatin (150 μM) to P3 FVB cochlear explants for 24 h, and number of outer hair cells per 160 μm of middle turn regions of the cochlea were counted by phalloidin staining, mean±SEM, P=*<0.05, P=***<0.001, compared to cisplatin alone by unpaired two-tailed Student's t-test. The initial molar ratio between the compounds tested was determined by the ratio given currently to cancer patients (dabrafenib at 150 mg twice daily plus trametinib at 2 mg once daily).

FIG. 10A shows data from mouse cochlear explants for a number of inhibitors (Mubritinib, SNS-314, and Crenolanib).

FIG. 10B shows data from mouse cochlear explants for a number of inhibitors (Mubritinib, SNS-314, and Crenolanib).

FIG. 11A, shows compounds protective effect in Zebrafish (Dabrafenib), Lateral line neuromasts of zebrafish were stained and number of hair cells per neuromast were counted. Cisplatin (CP): 400 μM. *, * and ***: P<0.05, 0.01, and 0.001 compared to CP alone.

FIG. 11B shows compounds protective effect in Zebrafish (Mubritinib) Lateral line neuromasts of zebrafish were stained and number of hair cells per neuromast were counted. Cisplatin (CP): 400 μM. *, **, and ***: P<0.05, 0.01, and 0.001 compared to CP alone.

FIG. 11C shows compounds protective effect in Zebrafish (Crenolanib). Lateral line neuromasts of zebrafish were stained and number of hair cells per neuromast were counted. Cisplatin (CP): 400 μM. *, **, and ***: P<0.05, 0.01, and 0.001 compared to CP alone.

FIG. 11D shows compounds protective effect in Zebrafish (SNS-314). Lateral line neuromasts of zebrafish were stained and number of hair cells per neuromast were counted. Cisplatin (CP): 400 μM. *, **, and ***: P<0.05, 0.01, and 0.001 compared to CP alone.

FIG. 12 shows the compounds Dabrafenib (a B-Raf kinase inhibitor, 30 nM) and AZD5438 (a CDK2 kinase inhibitor, 0.34 nM) protects against cisplatin, better than individual inhibitor, in mouse cochlear explants.

FIG. 13A shows protection against noise injury in mice by oral delivery of a combination of inhibitors (Dabrafenib 60 mg/kg×2 daily, AZD5438 35 mg/kg×2 daily).

FIG. 13B shows protection against noise injury in mice by oral delivery of a combination of inhibitors (Dabrafenib 60 mg/kg×2 daily, AZD5438 35 mg/kg×2 daily).

FIG. 13C shows protection against noise injury in mice by oral delivery of a combination of inhibitors (Dabrafenib 60 mg/kg×2 daily, AZD5438 35 mg/kg×2 daily).

FIG. 13D shows protection against noise injury in mice by oral delivery of a combination of inhibitors (Dabrafenib 60 mg/kg×2 daily, AZD5438 35 mg/kg×2 daily).

FIG. 14A shows the schedule of administration of dabrafenib and cisplatin in FVB mice. 30 mg/kg cisplatin was administered once on day one while 12 mg/kg dabrafenib was administered for three days, twice a day. Auditory testing was performed before treatment began and 21 days after cisplatin administration.

FIG. 14B shows ABR threshold shifts following protocol in FIG. 1A

FIG. 14C shows weight change over 21 days following protocol in FIG. 1A.

FIG. 14D shows Kaplan-Meier survival curves of mouse cohorts following protocol in (A). *P<0.05, **P<0.01, ***P<0.001 compared to cisplatin alone by two-way ANOVA with Bonferroni post hoc test.

FIG. 15A shows the schedule of administration of dabrafenib and cisplatin in a translational, multi-cycle treatment protocol using CBA/CaJ mice. Each cycle consisted of four days of treatment with 3 mg/kg cisplatin in the morning and 15, 3, or 0.6 mg/kg dabrafenib in the morning and evening. A 10-day recovery period followed the 4 days of treatment. This cycle was repeated a total of 3 times. Auditory testing occurred before treatment began, immediately after cycle 3 (day 42), and 4 months after cycle 3 (day 165).

FIG. 15B shows ABR threshold shifts recorded immediately after the completion of cycle 3 (day 42) in protocol shown in FIG. 15A.

FIG. 15C shows amplitudes of ABR wave 1 at 16 kHz from FIG. 15B.

FIG. 15D shows ABR threshold shifts of female.

FIG. 15E shows male mice recorded immediately after the completion of cycle 3.

FIG. 15F shows ABR threshold shifts recorded 4 months after the completion of cycle 3 (day 165). Carrier, cisplatin alone, 15 mg/kg dabrafenib alone, 3 mg/kg dabrafenib alone, 15 mg/kg dabrafenib plus cisplatin, 3 mg/kg dabrafenib plus cisplatin, and 0.6 mg/kg dabrafenib plus cisplatin. Data shown as means±SEM, *P<0.05, **P<0.01, ***P<0.001 compared to cisplatin alone by two-way ANOVA with Bonferroni post hoc test.

FIG. 16A shows DPOAE threshold shifts recorded immediately after the completion of cycle 3 (day 42) in protocol shown in FIG. 2A.

FIG. 16B shows DPOAE threshold shifts of female. Mice recorded immediately after the completion of cycle 3.

FIG. 16C shows DPOAE threshold shifts of male mice recorded immediately after the completion of cycle 3.

FIG. 16D shows DPOAE threshold shifts recorded 4 months after the completion of cycle 3 (day 165). Carrier, cisplatin alone, 15 mg/kg dabrafenib alone, 3 mg/kg dabrafenib alone, 15 mg/kg dabrafenib plus cisplatin, 3 mg/kg dabrafenib plus cisplatin, and 0.6 mg/kg dabrafenib plus cisplatin. Data shown as means±SEM, *P<0.05, **P<0.01, ***P<0.001 compared to cisplatin alone by two-way ANOVA with Bonferroni post hoc test.

FIG. 17A shows Representative EP measured from a CBA/CaJ mouse. The time of insertion into the endolymph and withdrawal is shown below the trace.

FIG. 17B shows Average EP measurements from mice before the treatment protocol in FIG. 17A began. Additionally, males and females are graphed individually.

FIG. 17C shows Average EP measurements of mice treated with carrier or cisplatin at different time points throughout protocol. Groups from left to right are as follows: Untreated mice before protocol began, carrier treated mice measured immediately after cycle 3 (day 42), cisplatin treated mice measured immediately after cycle 3, and cisplatin treated mice measured 4 months after cycle 3 (day 165). Data shown as means±SEM, all groups compared to one another by two-way ANOVA with Bonferroni post hoc test.

FIG. 18A shows representative myosin VI-stained confocal images of the 8-, 16-, and 32-kHz regions of the cochlea collected immediately after the completion of cycle 3 (day 42) of protocol shown in FIG. 15A.

FIG. 18B shows umber of outer hair cells per 160 μm at the 8-, 16, and 32-kHz regions of cochlea collected immediately after the completion of cycle 3.

FIG. 18C shows representative myosin VI-stained confocal images of the 8-, 16-, and 32-kHz regions of the cochlea collected 4 months after the completion of cycle 3 (day 165).

FIG. 18D shows number of outer hair cells per 160 μm at the 8-, 16, and 32-kHz regions of cochlea collected 4 months after the completion of cycle 3. Carrier, cisplatin alone, 15 mg/kg dabrafenib alone, 3 mg/kg dabrafenib alone, 15 mg/kg dabrafenib plus cisplatin, 3 mg/kg dabrafenib plus cisplatin, and 0.6 mg/kg dabrafenib plus cisplatin. Data shown as means±SEM, *P<0.05, **P<0.01, ***P<0.001 compared to cisplatin alone by two-way ANOVA with Bonferroni post hoc test. n=4-5,

FIG. 19A shows representative images of cochlear cryosections stained with DAPI (blue) and phosphorylated ERK (green) on day 4 of the protocol in FIG. 2A. Mice were sacrificed 45 minutes following the last cisplatin injection of cycle one. Total n=3 mice from each experimental group were tested.

FIG. 19B shows representative images of cochlear cryosections on day 32. Mice were sacrificed 45 minutes following the last cisplatin injection of cycle three. Experimental groups from left to right are as follows: carrier alone, cisplatin alone, 3.0 mg/kg dabrafenib alone, and 3.0 mg/kg dabrafenib+cisplatin. Total n=3 mice from each experimental group were tested.

FIG. 20A shows weight loss over the 42-day treatment protocol got Carrier, cisplatin alone, 15 mg/kg dabrafenib alone, 3 mg/kg dabrafenib alone, 15 mg/kg dabrafenib plus cisplatin, 3 mg/kg dabrafenib plus cisplatin, and 0.6 mg/kg dabrafenib plus cisplatin.

FIG. 20B shows Kaplan-Meier survival curves of mouse cohorts going to day 42 following protocol in FIG. 15A. Data shown as means±SEM, *P<0.05, **P<0.01, ***P<0.001 compared to cisplatin alone by two-way ANOVA with Bonferroni post hoc test.

FIG. 21A shows no differences in weight or survival was observed at Day 165. Mouse weights at day 165 of treatment protocol in FIG. 2A.

FIG. 21B shows Kaplan-Meier survival curves of mouse cohorts going to day 165. Carrier, cisplatin alone, 15 mg/kg dabrafenib alone, 3 mg/kg dabrafenib alone 15 mg/kg dabrafenib plus cisplatin (blue), 3 mg/kg dabrafenib plus cisplatin, and 0.6 mg/kg dabrafenib plus cisplatin.

FIG. 22A shows representative H&E and PAS images of the kidney at 20× magnification. Treatment groups from left to right are as follows: carrier alone, cisplatin alone, 3 mg/kg dabrafenib alone, 3 mg/kg dabrafenib plus cisplatin, 15 mg/kg dabrafenib alone, and 15 mg/kg dabrafenib plus cisplatin.

FIG. 22B shows Kidneys collected immediately after cycle 3.

FIG. 22C shows 4 months after cycle 3 were stained with H&E and PAS and scored blindly by an experienced pathologist. A score of 0 indicates no visible damage while a score of 4 indicates very severe damage.

FIG. 22D shows representative H&E and Masson's trichrome stained images of the liver at 20× magnification.

FIG. 22E shows Histology scores of liver samples collected immediately after cycle 3.

FIG. 22F shows 4 months after cycle 3 (165 days) blindly scored by experienced pathologist. 0=normal, 1=mild damage, 2=moderate damage, 3=severe damage, and 4=very severe (fulminant) damage. Data shown as means±SEM, all groups compared to one another by two-way ANOVA with Bonferroni post hoc test.

FIG. 23A shows representative images of kidneys dissected and stained at day 165 of the protocol. Representative H&E stained images of the kidney when dissected and stained at day 165 (4-months post cisplatin treatment). The 6 treatment groups that were analyzed are carrier alone, cisplatin alone, 15 mg/kg dabrafenib alone, 3 mg/kg dabrafenib alone, 15 mg/kg dabrafenib and cisplatin co-treatment, and 3 mg/kg dabrafenib and cisplatin co-treatment

FIG. 23B shows Representative PAS stained images of the kidney when dissected and stained at day 165.

FIG. 24A shows Representative images of livers dissected and stained at day 165 of the protocol.

FIG. 24B shows Representative images of livers dissected and stained at day 165 of the protocol.

### DETAILED DESCRIPTION

### Definitions

As used herein, the singular forms “a,” “an” and “the” include plural referents unless the content clearly dictates otherwise.

As used herein, the terms “about” and “approximately,” when used to modify a numeric value (e.g., “about 80 mg”) or one or more amounts specified in a range of numeric values (e.g., “about 80 mg to about 400 mg”), indicate the numeric value and functionally equivalent values, and reasonable deviations from the value known to or understood by those in the art, including persons of ordinary skill in the art or those skilled in the art. For example, values within ±10% or ±5% are within the intended meaning of a recited value. As such, “about 80 mg” is understood to encompass from “76 mg to 84 mg” or from “72 mg to 88 mg” as if written out each time. Thus, as used herein, the terms “about” and “approximately” intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art.

As used herein, the terms “administering” and “administration” refer to any method of providing a compound or composition to a subject. In some embodiments, the compound or composition is a pharmaceutical preparation. Such methods include, but are not limited to, oral administration, auricular administration, transdermal administration, administration by inhalation, nasal administration, topical administration, intraaural administration, intradural administration, intracerebral administration, sublingual administration and parenteral administration, including injectable such as intravenous administration, intra-arterial administration, intramuscular administration, and subcutaneous administration. Administration can be one-time, intermittent, or continuous. In some embodiments, a compound or composition of the invention is administered therapeutically, e.g., to treat an existing, diagnosed or suspected disease, disorder or condition. In some embodiments, a compound or composition of the invention is administered prophylactically, e.g., administered to prevent or inhibit progression of a disease, disorder or condition, or prevent the spread of a disease, disorder or condition.

As used herein, “effective amount” or “therapeutically effective amount” refers to an amount effective, at dosages and for periods of time as described in the dose regimens herein, to achieve a desired result in treatment of a disease, disorder, defect or condition for which one or more compounds or compositions of the invention are administered to a subject or to a patient. For example, and not by way of limitation, a “therapeutically effective amount” can refer to an amount of a cytoprotective drug compound or composition (e.g. a compound or composition comprising or consisting essentially of a cytoprotective drug), including but not limited to those disclosed herein, that is able to treat a disease, defect, disorder or condition when administered in accordance with the invention.

In some embodiments, “effective amount” or “therapeutically effective amount” refers to an amount effective a compound or composition, at dosages and for periods of time as described in the dose regimens herein or used by those in accordance with methods of the invention, to achieve a desired result or results in the treatment of one or more signs and/or symptoms of cisplatin-induced or noise-induced hearing loss. Symptoms include those described or referenced herein, for example, loss of hearing sensitivity, ringing in the ear, inner ear pain, and inner ear swelling. Therapeutically effective amounts include an amount that is sufficient to achieve at least one desired therapeutic result, or to have at least one effect on at least one undesired symptom associated with a disease, disorder, defect or condition, including but not limited to those referenced herein. The specific therapeutically effective dose level for any particular patient may be varied or depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors used in the medical arts in determining dose levels. For example, those in the art may start doses of a compound or composition at levels lower than those required to achieve the desired therapeutic effect and to gradually increase the dosage until the desired effect is achieved. In some embodiments, the result of treatment with an “effective amount” provides a durable result following treatment. In some embodiments, treatment with an effective amount is discontinued following a desired result. In some embodiments, treatment with an effective amount is continued following a desired result. In some embodiments, treatment with an effective amount is paused and then continued following a desired result. The doses disclosed herein are therapeutically effective amounts; however, the methods are not limited to those doses or dose amounts and include the use of other therapeutically effective amounts.

In some embodiments, a compound, composition or preparation can be administered in a “prophylactically effective amount”; that is, an amount effective for prevention of cisplatin-induced or noise-induced hearing loss. The compounds and compositions herein can be used to prevent, inhibit, or reduce the occurrence of, one or more signs or symptoms of noise-induced or cisplatin-induced hearing loss.

A “maintenance dose” or a “preventative dose” is a dose intended to be a therapeutically effective or a prophylactically effective dose. In some embodiments, the maintenance doses may be therapeutically effective doses and others may be sub-therapeutic doses. This disclosure includes dosing regimens and oral dosage formulations for the administration of maintenance doses of cytoprotective drugs. Such doses of cytoprotective drugs can be administered in cycles (e.g., a “first cycle”, a “second cycle”, a “third cycle”, etc.), during which each cycle a different amount of the cytoprotective drug is administered to a subject. For example, in some embodiments, the invention features a method of treating one or more signs and/or symptoms of noise-induced or cisplatin-induced hearing loss in a subject by (i) administering a loading-dose of a cytoprotective drug to the subject; and (ii) administering one or more maintenance doses of the cytoprotective drug to the subject, wherein each of the loading-dose and the maintenance doses are administered in an amount that together are sufficient to treat the one or more signs and/or symptoms of cisplatin-induced or noise-induced hearing loss. For example, the loading dose can be administered by injection (e.g., subcutaneously) or orally followed by maintenance dosing administered orally, intravenously, nasally, subcutaneously, transdermally, or by direct administration to the inner ear. In some embodiments, the loading-dose is administered orally. In some embodiments, the loading-dose is administered by direct administration to the inner ear by the methods described herein. In some embodiments, the introductory or loading dose is about 15 to 30 mg/kg administered daily and the maintenance dose is about 0.6 to about 15 mg/kg administered about once a week. In some embodiments, the dose for the induction phase, e.g., the introductory or loading dose, is a dose of 15 to 30 mg/kg administered at least once per day and the dose for the maintenance phase is about 0.6 to 15 mg/kg once a week.

The term “pharmaceutical formulation” refers to a preparation which is in such form as to permit the biological activity of an active ingredient contained therein to be effective, and which does not contain additional components that are unacceptably toxic to a subject to which the formulation would be administered. A “pharmaceutical composition” refers to a mixture of substances suitable for administering to a subject that includes one or more active ingredients or pharmaceutical agents (e.g., one or more cytoprotective drugs). For example, a pharmaceutical composition may include one or more compounds of the invention and a sterile aqueous solution or a pharmaceutically acceptable carrier.

A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, a buffer, excipient, stabilizer, or preservative.

As used herein, the term “prevent” or “preventing” refers to precluding, averting, obviating, forestalling, stopping, or hindering something from happening. It is understood that where reduce, inhibit or prevent are used herein, unless specifically indicated otherwise, the use of the other two words is also expressly disclosed, and that they refer to reducing, inhibiting or preventing in whole or in part for the therapeutic and/or prophylactic purposes described herein.

As used herein, the term “subject” or the like, refers to any animal, including humans, domestic and farm animals, and zoo, wild animal park, sports, or pet animals, such as dogs, horses, cats, fish (e.g., zebrafish), sheep, pigs, cows, etc. In some instances, however, a patient may refer to a subject afflicted with specific a condition, disease, defect, syndrome or disorder. The terms “subject” and “patient” include human and veterinary subjects. The preferred animal herein is a human, including adults, children, and the elderly. The term “subject” does not denote a particular age or sex. In some embodiments, subjects may include animals used in scientific experiments (e.g. mice, rats, rabbits, sheep, goats, or other laboratory subjects). In other embodiments, the term “subjects” may exclude one or more or all animals used in scientific experiments, and a method may specify that is does not include one or more or all animals used in scientific experiments. In some embodiments, subjects may include non-animals and other things used in scientific experiments (e.g., fruit flies, cells, cell cultures, tissues, organs, 3D tissue culture (such as organs-on-a-chip). In other embodiments, the term “subjects” may exclude one or more or all non-animals or other things used in scientific experiments and a method may specify that is does not include one or more or all non-animals or other things used in scientific experiments. In some embodiments of the disclosed methods, a subject has been diagnosed with a need for treatment of one or more signs and/or symptoms associated with cisplatin-induced or noise-induced hearing loss.

As used herein, “treatment”, “treat” and “treating”, refers to an attempt to alter the course of the individual, tissue or cell being treated, and can be performed either for prophylaxis or during the course of clinical pathology. Thus, the term “treatment” includes the management or care of a subject, or of a patient, with the intent to cure, ameliorate, stabilize, or prevent a disease, disorder, defect or condition. This term includes active treatment, that is, treatment directed specifically toward the improvement of a disease, disorder, defect or condition, and also includes causal treatment, that is, treatment directed toward removal of the cause of the associated disease, disorder, defect or condition. In addition, this term includes palliative treatment, that is, treatment designed for the relief of symptoms rather than the curing of the disease, disorder, defect or condition; preventative treatment, that is, treatment directed to minimizing or partially or completely inhibiting the development of the associated disease, disorder, defect or condition; and supportive treatment, that is, treatment employed to supplement another specific therapy directed toward the improvement of the associated disease, disorder, defect or condition. In various aspects, the term covers any treatment of a subject, including a mammal (e.g., a human), and includes: (i) preventing a disease, disorder or condition from occurring in a subject that can be predisposed to the disease, disorder or condition but has not yet been diagnosed as having it; (ii) inhibiting a disease, disorder or condition, i.e., arresting or slowing its development; or (iii) relieving a disease, disorder or condition, i.e., causing regression of the disease, disorder or condition. In some embodiments, the subject is a human. Treatment does not necessarily imply that a subject or patient is treated until total recovery following treatment, or that treatment of a subject or patient results in total recovery. Accordingly, “treatment” may also include maintaining or promoting a complete or partial state of remission in a subject or patient, or an alleviation or relief from symptoms of the disease, disorder, defect of conditions being treated, whether in whole or in part, temporary or permanent. The term “preventing” means preventing in whole or in part or ameliorating or controlling. The term “ameliorate,” “ameliorating” and “amelioration” and the like refer an improvement in one or more signs or symptoms of noise-induced or cisplatin-induced hearing loss, a reduction in the severity of one or more signs or symptoms of noise-induced or cisplatin-induced hearing loss, or an inhibition of progression or worsening of one or more signs or symptoms of noise-induced or cisplatin-induced hearing loss or disorder.

In some embodiments, compounds, compositions, methods, and kits of the invention are used in the treatment of a subject or a patient for one or more signs and/or symptoms of noise-induced or cisplatin-induced hearing loss.

In some embodiments, compounds, compositions, methods, and kits of the invention are used alleviation of one or more signs or symptoms of, or diminishment of one or more direct or indirect pathological consequences of noise-induced or cisplatin-induced hearing loss.

The widely used chemotherapy cisplatin causes permanent hearing loss in 40-60% of cancer patients. Dabrafenib, an FDA-approved BRAF kinase inhibitor and anticancer drug, showed protective effects in a clinically relevant multi-dose cisplatin mouse model. The protective effects of dabrafenib, given orally twice daily with cisplatin, were determined by functional hearing tests and cochlear outer hair cells counts. Toxicity of the drugs co-treatment was evaluated, and levels of pERK were measured. Dabrafenib, in dose of 3 mg/kg/bw, twice daily, in mice, was determined to be the minimum effective dose and it is equivalent to one tenth of the daily FDA-approved dose for human cancer treatment. The levels of hearing protection acquired, 20-25 dB at the three frequencies tested, in both female and male mice, persisted for four months after completion of treatments. Moreover, dabrafenib exhibited a good in vivo therapeutic index (>25), hearing protection in two different mouse strains, and diminished cisplatin-induced weight loss.

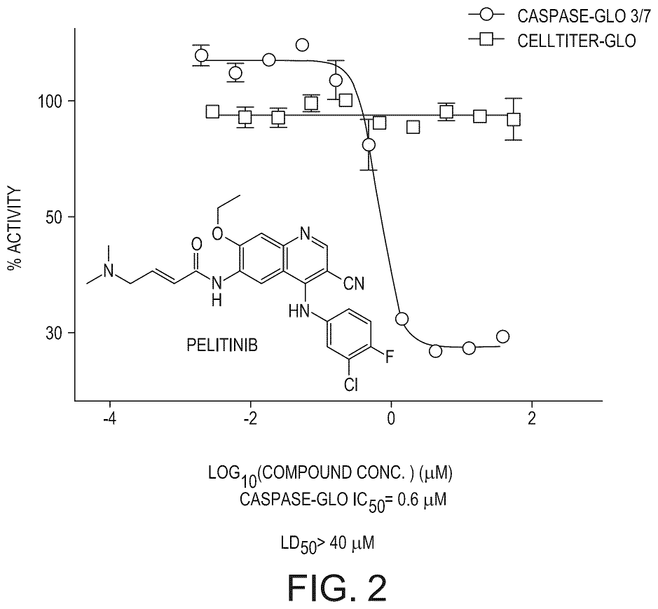

Human cancer patients typically receive a week of daily cisplatin infusions in cycles spaced a few weeks apart. Employing a clinically relevant cisplatin protocol and three 1:5 dilutions of the drug dabrafenib (15, 3, 0.6 mg/kg), we conclude that dabrafenib has an average protection of 19 dB at 8 kHz, 25 dB at 16 kHz, and 34 dB at 32 kHz, after cisplatin treatment with a low dose 3 mg/kg twice daily (FIG. 2). Significantly, the dose of 3 mg/kg/bw dabrafenib, twice daily, was found to be as effective as the 15 mg/kg/bw and is approximately one tenth of the equivalent dabrafenib human dose given to cancer patients. 15 and 3 mg/kg dabrafenib exhibited the same hearing protection with no statistically significant difference between the groups. Thus, 3 mg/kg was determined to be the minimal effective dose in this model. The lowest dose tested of 0.6 mg/kg dabrafenib, which is equivalent to one fiftieth of the human equivalent dose, still demonstrated protection of 12 dB at 8 kHz, 15 dB at 16 kHz, and 20 dB at 32 kHz is still detected, yet it is not as effective as 3 or 15 mg/kg dabrafenib. The multi-dose protocol demonstrated a therapeutic window of at least 25 for dabrafenib in vivo. Protection was observed with a dose as high as 15 mg/kg and as low as 0.6 mg/kg. Higher doses of dabrafenib were not tested, however, previous data obtained from the single, high dose cisplatin protocol demonstrated 100 mg/kg dabrafenib daily was well tolerated. A wide therapeutic index is important for the clinical application of dabrafenib to give clinicians flexibility with dosage without toxicity to the patient.

Protection from weight-loss in the cisplatin and dabrafenib co-treated groups, employing either the single-dose protocol or the multi-dose regimen, is an unexpected and exciting phenomenon in our studies. Dabrafenib significantly reduces the weight loss typically seen in mice during cisplatin treatment and thus helps maintain the general well-being of the animals.

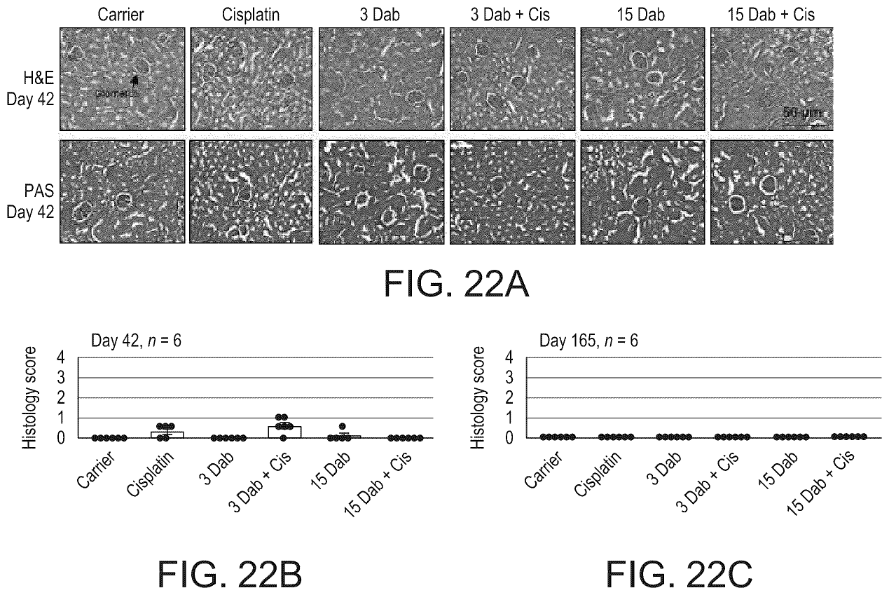

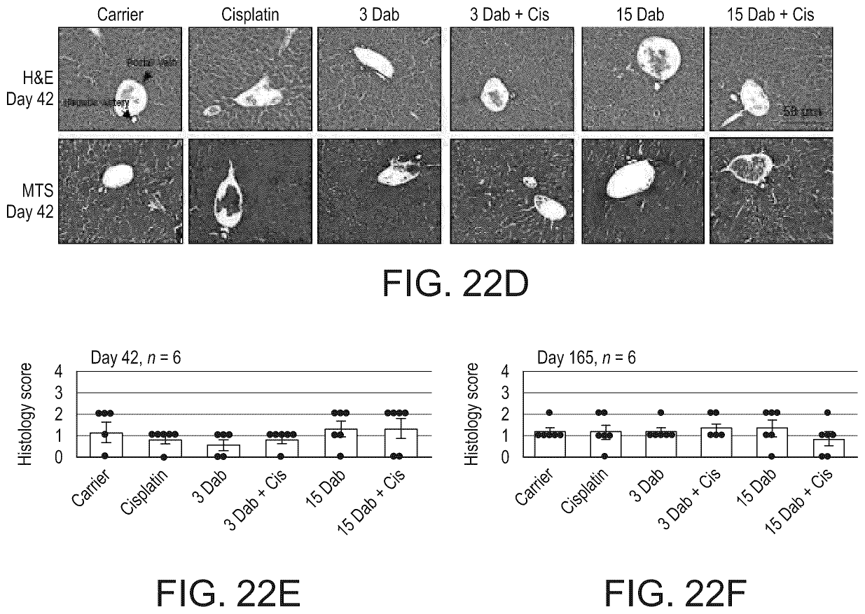

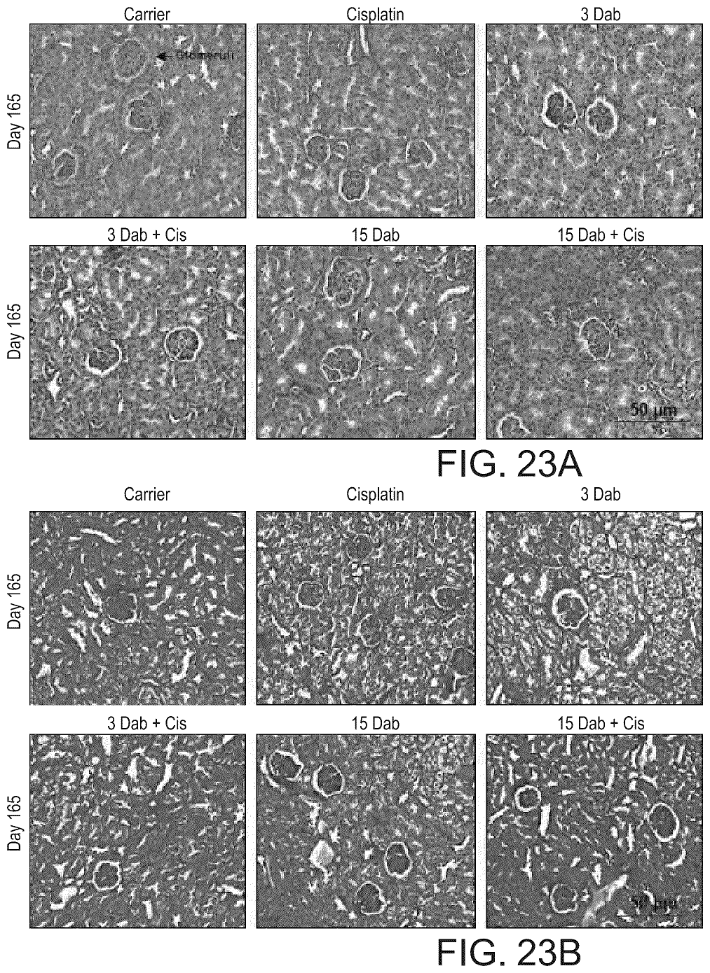

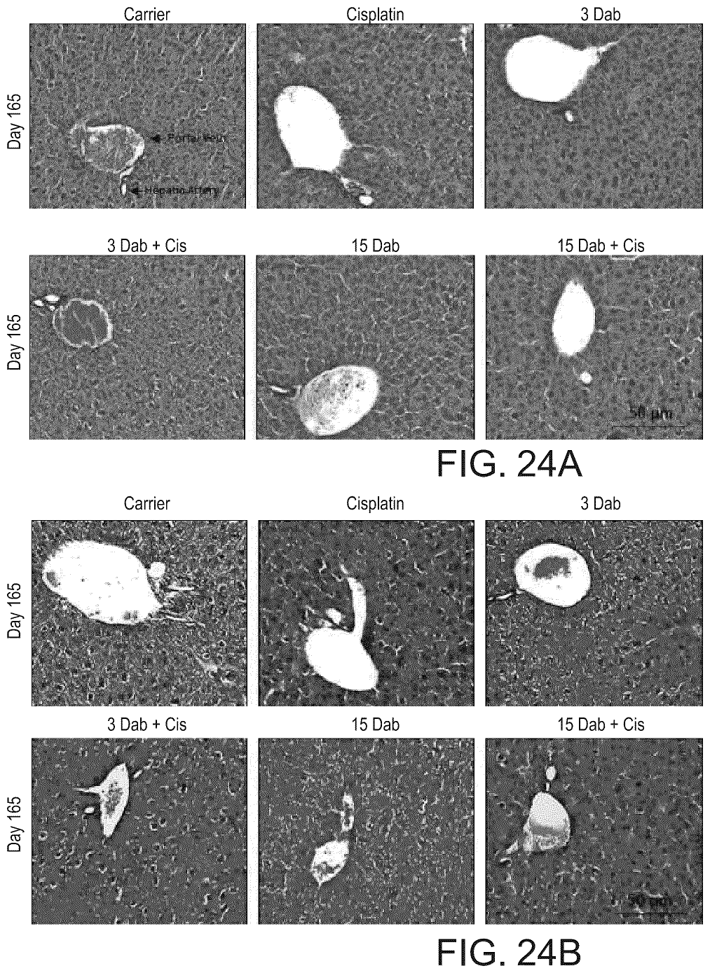

Toxicity of dabrafenib with cisplatin treatment was tested in this study in the kidney and livers of the treated animals. While combining two drugs together could typically be expected to present systemic toxicity issues; the inventors discovered that the combination of dabrafenib and cisplatin was not toxic to major organs that can be damaged from cisplatin. These organs were chosen as it is known that, in addition to the ear, cisplatin accumulates and can cause damage in these tissues. As shown in FIG. 22, FIG. 23 and FIG. 24, no significant damage was recorded by H&E, PAS, and Masson's Trichrome staining in the kidneys or livers of the mice at days 42 and 165 with the co-treatments. Dabrafenib alone, being an FDA-approved drug, was not expected to cause significant damage to the kidneys and livers of the mice in the doses tested in this study, but the toxicity and ototoxicity of the co-treatments were unknown. This demonstration of no significant toxicity or ototoxicity of the drug co-treatments is vital for future clinical trials and is a surprising effect given the typical toxicity associated with combining cisplatin and other drugs.

Cisplatin has been shown to accumulate in the inner ear by the Breglio et al. study and may cause long-term hearing loss and possible reduced protection when drug administration does not continue after the cessation of cisplatin treatment. For that reason, it is important to test if dabrafenib will protect not only at day 42 when the cisplatin cycles are completed, but also at longer time points, such as four months after the treatments. The data shows that dabrafenib co-treated mice have significantly better hearing ability compared to cisplatin alone mice. The hearing protection is sustained for up to 4 months following the end of cisplatin treatment, which indicates the protection dabrafenib offers from cisplatin ototoxicity is stable and not acute. Mice only need to be treated with dabrafenib while cisplatin is administered and more treatments following the cessation of cisplatin are not required to confer protection. This limits the amount of drug patients would need to receive to get optimal hearing protection from dabrafenib.

It has now been found that inhibitors of EGFR and proteins downstream or associated therewith protect hair cells damaged by cisplatin, antibiotics, noise, aging or other ototoxic insults. Accordingly, this invention provides compositions and methods for the prevention and treatment of hearing loss using an inhibitor of EGFR. Ideally, the inventive methods prophylactically or therapeutically treat an animal, preferably a mammal (e.g., a human), for at least one disorder associated with loss or damage of sensory hair cells, e.g., disorders of the ear associated with damage of sensory hair cells (such as hearing loss or balance disorders). Inventive methods also are useful in maintaining a level of sensory perception, i.e., controlling the loss of perception of environmental stimuli caused by, for instance, the aging process or ototoxic agents. Inhibitors of EGFR and proteins downstream or associated therewith provide a therapeutic effect.

An inhibitor of this invention can selectively decrease or block the expression of an EGFR signaling protein (i.e., transcription or translation of the protein), decrease or block the activity of an EGFR signaling protein (i.e., binding to ligands, tyrosine kinase activity, phosphorylation, protein-protein interactions, and/or downstream signaling), decrease or block the biological effect(s) of an EGFR signaling protein, and/or modify half-life or subcellular localization (membrane versus cytoplasmic or nuclear localization, internalization, and recycling) of an EGFR signaling protein. In particular, an inhibitor of EGFR signaling is an active agent that selectively decreases or blocks one or more of the following: transcription or translation, ligand binding, phosphorylation, multimerization, tyrosine kinase activity, internalization, and/or translocation into the nucleus.

Ideally, EGFR signaling is completely blocked, or is reduced by at least 10%, at least 20%, at least 30%, at least 40%, at least 50%, at least 60%, at least 70%, at least 75%, at least 80%, at least 85%, at least 90%, at least 92.5%, at least 95%, at least 97%, at least 98%, at least 98.5%, at least 99%, at least 99.25%, at least 99.5%, or at least 99.75% by the inhibitor of EGFR signaling inhibitor as compared to normal physiologic levels.

The inhibitor of EGFR signaling of this invention typically has a half maximal (50%) inhibitory concentration (IC₅₀) in the range of 1 μM to 100 μM. Preferably, the inhibitor of EGFR signaling has an IC₅₀ value of less than 10 μM, less than 5 μM, less than 1 μM, or less than 100 nM. Moreover, in some embodiments, the inhibitor of EGFR signaling is specific/selective for one or more of the EGFR signaling proteins of interest and fails to inhibit or inhibits to a substantially lesser degree other non-EGFR pathway proteins. In this respect, it is preferable that the inhibitor of EGFR signaling is a selective inhibitor of EGFR signaling. Preferably, selectivity is for one, two, three or four EGFR signaling proteins and fails to inhibit or inhibits to a substantially lesser degree other non-EGFR pathway proteins. By way of illustration, an inhibitor can be a dual EGFR and ERBB2 inhibitor, both of which are EGFR signaling proteins. Methods for assessing the selectively of inhibitors are known in the art and can be based upon anv assay including, but not limited to the determination of the IC₅₀, the binding affinity of the inhibitor (i.e., Kᵢ), and/or the half maximal effective concentration (EC₅₀) of the inhibitor for EGFR signaling protein of interest as compared to another protein (comparative protein). In particular embodiments, a selective inhibitor of EGFR signaling is an inhibitor that has an IC₅₀ value for an EGFR signaling protein of interest that is at least twice or, more desirably, at least three, four, five, or six times lower than the corresponding IC₅₀ value for a comparative protein. Most desirably, a selective inhibitor of EGFR signaling has an IC₅₀ value for an EGFR signaling protein which is at least one order of magnitude or at least two orders of magnitude lower than the IC₅₀ value for a comparative protein.

Sensory Perception. The invention provides for the modulation of sensory perception in an animal by administering to the inner ear an inhibitor of EGFR signaling as an otoprotective agent. By “modulating sensory perception” it means achieving, at least in part, the ability to recognize and adapt to environmental changes. In terms of sensory hair cell function, modulation in sensory perception is associated with the generation or protection of sensory hair cells that convert mechanical stimuli in the inner ear into neural impulses, which are then processed in the brain such that an animal is aware of environmental change, e.g., sound, language, or body/head position. Sensory hair cells are preferably generated in the organ of Corti and/or vestibular apparatus. In the context of prophylaxis, sensory hair cells, which would otherwise be initially or further damaged or lost due to, e.g., ototoxic agents, are protected from damage or loss by the administration of an inhibitor of EGFR signaling and optionally an otoprotective agent.

A change in the ability of a subject to detect sound is readily accomplished through administration of simple hearing tests, such as a tone test commonly administered by an audiologist. In most mammals, a reaction to different frequencies indicates a change in sensory perception. In humans, comprehension of language also is appropriate. For example, it is possible for a subject to hear while being unable to understand speech. A change in perception is indicated by the ability to distinguish different types of acoustic stimuli, such as differentiating language from background noise, and by understanding speech. Speech threshold and discrimination tests are useful for such evaluations.

Evaluation of changes in balance, motion awareness, and/or timing of response to motion stimuli also is achieved using a variety of techniques. Vestibular function also can be measured by comparing the magnitude of response to motion stimulus (gain) or timing of initiation of response (phase). Animals can be tested for Vestibulo-Ocular Reflex (VOR) gain and phase using scleral search coils to evaluate improvements in sensory perception. Electronystagmography (ENG) records eye movements in response to stimuli such as, for instance, moving or flashing lights, body repositioning, fluid movement inside the semicircular canals, and the like. Evaluation of balance during movement using a rotating chair or moving platform is also useful in this respect.

To detect a change in sensory perception, a baseline value is recorded prior to the inventive method using any appropriate sensory test. A subject is reevaluated at an appropriate time period following the inventive method (e.g., 1 hour, 6 hours, 12 hours, 18 hours, 1 day, 3 days, 5 days, 7 days, 14 days, 21 days, 28 days, 2 months, 3 months or more following the inventive method), the results of which are compared to baseline results to determine a change in sensory perception.

Method of Prevention or Treatment. The inventive method promotes the protection and/or generation of sensory hair cells that allow perception of stimuli. Accordingly, this invention provides a method for the prevention, treatment, control, amelioration, or reduction of risk of hearing impairments, loss, and disorders by administering to a subject in need of treatment an inhibitor of EGFR signaling and/or one or more otoprotective/regenerative agents. Ideally, the inventive method prophylactically or therapeutically treats an animal for at least one disorder associated with loss, damage, absence of sensory hair cells, such as hearing loss and balance disorders. Hearing loss can be caused by damage of hair cells of the organ of Corti due to bacterial or viral infection, heredity, physical injury, acoustic trauma, ototoxic drugs (e.g., aminoglycoside antibiotic or cisplatin) and the like. While hearing loss is easily identified, balance disorders manifest in a broad variety of complications easily attributable to other ailments. Symptoms of a balance disorder include disorientation, dizziness, vertigo, nausea, blurred vision, clumsiness, and frequent falls. Balance disorders treated by the inventive method preferably involve a peripheral vestibular disorder (i.e., a disturbance in the vestibular apparatus) involving dysfunctional translation of mechanical stimuli into neural impulses due to damage or lack of sensory hair cells.

In one aspect, methods of protecting against or preventing hearing loss or impairment are provided. In accordance with such methods, a subject in need of treatment is administered an effective amount of inhibitor of EGFR signaling. In some embodiments, the inhibitor of EGFR signaling inhibits the expression or activity of EGFR, a Ras/Raf/MEK/ERK/MAPK protein, a JAK/STAT protein, a PI3K/AKT/mTOR protein, a NCK-PAK-JNK protein, a PLC-DAG-PKC protein, or a cell cycle-associated protein kinase associated with or downstream of EGFR. In other embodiments, prevention of hearing loss is achieved by administering to a subject in need of treatment an inhibitor of a cell cycle-associated protein kinase associated with or downstream of EGFR. In certain embodiments, prevention of hearing loss is achieved by administering to a subject in need of treatment an inhibitor of Her-2, Aurora kinase, B-Raf, or PDGFR expression or activity. The inhibitor of EGFR signaling can be administered alone or in combination with one or more otoprotective agents. The term “otoprotective agent” refers to an agent that reduces or prevents noise-induced hearing loss, chemically-induced hearing loss, or age-induced hearing impairment or otherwise protects against hearing impairment. Examples of otoprotective agents include, but are not limited to, PARP-1 inhibitors; pirenzepine LS-75, otenzepad, AQ-RA741, viramune, BIBN 99, DIBD, telenzepine (see US 2011/0263574); methionine (see U.S. Pat. No. 7,071,230); IGF-1, FGF-2, aspirin, reduced glutathione, N-methyl-(D)-glucaminedithiocarbamate, and iron chelators such as tartrate and maleate. See also US 2005/0101534 for additional otoprotective agents.

Protection against and prevention or treatment of hearing loss or impairment can be in the context of conditions including, but not limited to, tinnitus, ringing, Presbyacusis, auditory neuropathy, acoustic trauma, acoustic neuroma, Pendred syndrome, Usher syndrome, Wardenburg syndrome, non-syndromic sensorineural deafness, otitis media, otosclerosis, Meniere's disease, ototoxicity, labyrinthitis, and hearing impairments caused by infection (i.e., measles, mumps, or meningitis), medicines such as antibiotics, and some cancer treatments (i.e., chemotherapy and radiation therapy).

In certain embodiments, the hearing impairment is drug-induced. In a still further aspect, the drug is a chemotherapeutic agent. More specifically, the drug is a platinum-based chemotherapeutic agent such as carboplatin, cisplatin, transplatin, nedaplatin, oxaliplatin, picoplatin, satraplatin, transplatin, and triplatin, or a pharmaceutically acceptable salt thereof. In a particular embodiment, the platinum-based chemotherapeutic agent is cisplatin, or a pharmaceutically acceptable salt thereof. In yet a further embodiment, the drug is an antibiotic, including, but not limited to, daunorubicin, doxorubicin, epirubicin, idarubicin, actinomycin-D, bleomycin, mitomycin-C, amikacin, apramycin, arbekacin, astromicin, bekanamycin, dibekacin, framycetin, gentamicin, hygromycin B, isepamicin, kanamycin, neomycin, netilmicin, paromomycin, rhodostreptomycin, ribostamycin, sisomicin, spectinomycin, streptomycin, tobramycin, and verdamicin, or a pharmaceutically acceptable salt thereof.

In a further aspect, the hearing impairment is age-related, noise-induced or a balance or orientation-related disorder. Examples of balance disorders include, but are not limited to, induced or spontaneous vertigo, dysequilibrium, increased susceptibility to motion sickness, nausea, vomiting, ataxia, labyrinthitis, oscillopsia, nystagmus, syncope, lightheadedness, dizziness, increased falling, difficulty walking at night, Meniere's disease, and difficulty in visual tracking and processing. Further, the noise-induced hearing loss may be temporary or permanent.

More than one billion teens and young adults worldwide are at risk of hearing loss from exposure to loud music, as recently reported by the World Health Organization. Many other noise exposures, including occupational settings and consumer-operated devices, also cause noise-induced hearing loss, which is among the most common physical complaints, and which detracts significantly from the ability to converse, communicate, and participate in everyday life (thus reducing general quality of life of the individual and the family). Acute or chronic acoustic overexposure has put more than 40 million US workers at risk of permanent hearing loss (Kopke, et al. (2007) Hear. Res. 226:114-125).

Traumatic brain injury (TBI) and blast-associated injury occur most frequently in military situations where blast exposure cannot be predicted, trauma intensity exceeds the effectiveness of protective devices, or protective devices are not available. TBI is often accompanied by a diverse range of disruption or damage to the auditory sensory system, which is highly vulnerable to blast injury. Extreme physical blast force can cause damage of various types to the peripheral auditory system, including rupture of the tympanic membrane (TM, eardrum), fracture of the middle ear bones, dislocation of sensory hair cells from the basilar membrane, and loss of spiral ganglia that innervate hair cells. In human studies of blast injury, approximately 17-29% of cases involve severe TM rupture, while 33-78% involve moderate to severe sensorineural hearing loss (hair cell and ganglion loss). Therefore, TBI and blast injury are a common, although extreme, cause of hearing loss.

Biological protection of hearing is more promising than currently available mechanical protective devices. Hearing aids are frequently problematic because of their high cost and their many technical issues. Ideally, service men and women could take protective drugs before entering high-risk or high-noise settings and would then be protected from noise injury with no effect on performance. To date, there are no FDA-approved drugs for protection against noise- and TBI-associated hearing loss.

In accordance with the methods of this invention, the inhibitor of EGFR signaling can be administered locally, e.g., to the inner ear of the subject by the methods described herein. In some embodiments, the inhibitor of EGFR signaling can be administered systemically. Further, the inhibitor of EGFR signaling can be administered via injection into one or more of the scala tympani, cochlear duct, scala vestibule of the cochlea, into the auditory nerve trunk in the internal auditory meatus, or into the middle ear space across the transtympanic membrane/ear drum. Moreover, when used in combination, the EGFR signaling can be administered via the same or different routes.

In various aspects, the disclosed molecules can be used in combination with one or more other drugs in the treatment, prevention, control, amelioration, or reduction of risk of hearing impairments and disorders for which disclosed molecules or the other drugs can have utility, where the combination of the drugs together are safer or more effective than either drug alone. Such other drug(s) can be administered, by a route and in an amount commonly used therefor, contemporaneously or sequentially with a compound of the present invention. When a molecule of the present invention is used contemporaneously with one or more other drugs, a pharmaceutical composition in unit dosage form containing such other drugs and a disclosed compound is preferred. However, the combination therapy can also include therapies in which a disclosed molecule and one or more other drugs are administered on different overlapping schedules. It is also contemplated that when used in combination with one or more other active ingredients, the disclosed molecules and the other active ingredients can be used in lower doses than when each is used singly.

The methods herein are useful in the prevention or treatment of both acute and persistent, progressive disorders associated with lack of or damage to functional sensory hair cells. For acute ailments, the drugs herein can be administered using a single application or multiple applications within a short time period. For persistent diseases, such as hearing loss, or disorders stemming from a massive loss of sensory hair cells, numerous rounds of administration of the drugs herein may be necessary to realize a therapeutic effect.

Where appropriate, following treatment, the subject (e.g., human or other animal) can be tested for an improvement in hearing or in other symptoms related to hearing disorders. Subjects benefiting from treatment include those at risk of hearing hair cell loss. For example, a subject having or at risk for developing a hearing loss can hear less well than the average subject (e.g., an average human being), or less well than a subject before experiencing the hearing loss. For example, hearing can be diminished by at least 5%, 10%, 30%, 50% or more. Methods for measuring hearing include pure tone audiometry, air conduction, and bone conduction tests. These exams measure the limits of loudness (intensity) and pitch (frequency) that a human can hear. Hearing tests in humans include behavioral observation audiometry (for infants to seven months), visual reinforcement orientation audiometry (for children 7 months to 3 years) and play audiometry for children older than 3 years. Oto-acoustic emission testing can be used to test the functioning of the cochlear hair cells, and electro-cochleography provides information about the functioning of the cochlea and the first part of the nerve pathway to the brain. In various aspects, treatment can be continued with or without modification or can be stopped.

### Reducing Cisplatin-Induced Chemotherapy Side Effects.

In another embodiment, this disclosure provides for a method of reducing a condition or disorder resulting from a chemotherapeutic side effect or a radiotherapy side effect. The chemotherapeutic agent causing the side effects can be cisplatin. One or more of the B-raf inhibitors described herein (e.g., dabrafenib) may be used for treating and/or preventing (i.e., reducing the likelihood of occurrence of) a chemotherapeutic side effect or a radiotherapy side effect. Chemotherapeutic side effects include but are not limited to gastrointestinal toxicity (e.g., nausea, vomiting, constipation, anorexia, diarrhea), peripheral neuropathy, fatigue, malaise, low physical activity, hematological toxicity (e.g., anemia), hepatotoxicity, alopecia (hair loss), pain, infection, mucositis, fluid retention, dermatological toxicity (e.g., rashes, dermatitis, hyperpigmentation, hearing loss, weight loss, kidney damage, urticaria, photosensitivity, nail changes), mouth (e.g., oral mucositis), or gum or throat problems. As described herein, methods for treating or preventing (i.e., reducing the likelihood of occurrence of) chemotherapy side effects can be performed by administering the B-raf inhibitor (e.g., dabrafenib) during the off-chemotherapy or off-radiotherapy time interval as a first cycle, or after the chemotherapy or radiotherapy treatment regimen has been completed, as a first cycle.

In one embodiment, the chemotherapy side effect may include one or more of weight loss, endocrine change(s) (e.g., hormone imbalance, change in hormone signaling), and change(s) in body composition (e.g., body mass). In certain embodiments, the chemotherapy side effects can include decreased or reduced ability of the subject to be physically active, as indicated by decreased or diminished expenditure of energy than would be observed in a subject who did not receive the chemotherapy.

In one embodiments, a B-raf inhibitor (e.g., dabrafenib) described herein may be used in the methods as provided herein for ameliorating chronic or long term side effects resulting from chemotherapy. Chronic toxic side effects typically result from multiple exposures to or administrations of a chemotherapy or radiotherapy over a longer period of time. Certain toxic effects appear long after treatment (also called late toxic effects) and result from damage to an organ or system by the therapy. Chronic and/or late toxic side effects that occur in subjects who received chemotherapy or radiation therapy can include hearing loss.

In particular, it is contemplated that chemotherapeutic drugs (e.g., cisplatin) that have a high level of toxicity will benefit from administration of chemotherapeutic drugs in combination with a B-raf inhibitor (e.g., dabrafenib) as described herein. Administration of cisplatin and dabrafenib, for example, will result in decreased toxicity of the drug (e.g., to non-target tissues). Thus, the combination of the chemotherapeutic drug (e.g., cisplatin) with the B-raf inhibitor (e.g., dabrafenib) may increase the therapeutic index of the chemotherapeutic drug by both reducing side effects and improving efficacy of the drug and may result in a formulation of the chemotherapeutic drug that has an acceptable therapeutic index and can be pursued/approved for use in humans having the target disease.

Routes of Administration. One skilled in the art will appreciate that suitable methods of administering a drug to the inner ear are available. Although more than one route can be used to administer a particular drug, a particular route can provide a more immediate and more effective reaction than another route. Accordingly, the described routes of administration are merely exemplary and are in no way limiting.

Without being bound by theory, the compounds of this disclosure are formulated to reach the sensory epithelium of the inner ear. The most direct routes of administration, therefore, entail surgical procedures which allow access to the interior of the structures of the inner ear. Inoculation via cochleostomy allows administration of a drug directly to the regions of the inner ear associated with hearing. Cochleostomy involves drilling a hole through the cochlear wall, e.g., in the otic capsule below the stapedial artery as described in Kawamoto, et al. ((2001) Molecular Therapy 4(6):575-585), and release of a pharmaceutical composition containing the drug. Administration to the endolymphatic compartment is particularly useful for administering a drug to the areas of the inner ear responsible for hearing. Alternatively, a drug can be administered to the semicircular canals via canalostomy. Canalostomy provides for drug delivery to the vestibular system and the cochlea, whereas cochleostomy does not provide as efficient drug delivery in the vestibular space. The risk of damage to cochlear function is reduced using canalostomy in as much as direct injection into the cochlear space can result in mechanical damage to hair cells (Kawamoto, et al., supra). Administration procedures also can be performed under fluid (e.g., artificial perilymph), which can include factors to alleviate side effects of treatment or the administration procedure, such as apoptosis inhibitors or anti-inflammatories.

Another direct route of administration to the inner ear is through the round window, either by injection or topical application to the round window. Administration via the round window is especially preferred for delivering a drug to the perilymphatic space.

In some embodiments, the compounds of this disclosure can be administered orally for systemic administration. In some embodiments, the compounds if this disclosure can be administered in an ear drop formulation, for local administration of the drug. In some embodiments, the drugs of this disclosure can be administered intranasally, interperitonally, intravitreally, sublingually, topically, or by suppository.

A drug can be present in a pharmaceutical composition for administration to the inner ear. In certain cases, it may be appropriate to administer multiple applications and/or employ multiple routes, e.g., canalostomy and cochleostomy, to ensure sufficient exposure of supporting cells to the drug.

A drug can be present in or on a device that allows controlled or sustained release of the drug, such as a sponge, meshwork, mechanical reservoir or pump, or mechanical implant. For example, a biocompatible sponge or gelform soaked in a pharmaceutical composition containing the drug is placed adjacent to the round window, through which the drug permeates to reach the cochlea (as described in Jero, et al., supra). Mini-osmotic pumps provide sustained release of a drug over extended periods of time (e.g., five to seven days), allowing small volumes of composition containing the drug to be administered, which can prevent mechanical damage to endogenous sensory cells. The drug also can be administered in the form of sustained-release formulations (see, e.g., U.S. Pat. No. 5,378,475) containing, for example, gelatin, chondroitin sulfate, a polyphosphoester, such as bis-2-hydroxyethyl-terephthalate (BHET), or a polylactic-glycolic acid.

Alternatively, the drug can be administered parenterally, intramuscularly, intravenously, orally or intraperitoneally. Pharmaceutically acceptable carriers for compositions are well-known to those of ordinary skill in the art (see Pharmaceutics and Pharmacy Practice, (1982) J.B. Lippincott Co., Philadelphia, PA, Banker and Chalmers, eds., pages 238-250; ASHP Handbook on Injectable Drugs (1986) Toissel, 4^{th} ed., pages 622-630).

Dosage. The dose of a drug administered to an animal, particularly a human, in accordance with the invention should be sufficient to affect the desired response in the animal over a reasonable time frame. One skilled in the art will recognize that dosage will depend upon a variety of factors, including the age, species, location of damaged sensory epithelia, the pathology in question (if any), and condition or disease state. Dosage also depends on the inhibitor of EGFR signaling and/or cell cycle-associated protein kinase inhibitor, and the amount of sensory epithelium to be transduced. The size of the dose also will be determined by the route, timing, and frequency of administration and the existence, nature, and extent of any adverse side effects that might accompany the administration of a particular drug and the desired physiological effect. It will be appreciated by one of ordinary skill in the art that various conditions or disease states, in particular, chronic conditions or disease states, may require prolonged treatment involving multiple administrations. One skilled in the art can extrapolate the dose conversion between animals and humans. See e.g. A. Nair, A Simple Practice Guide for Dose Conversion Between Animals and Humans. J. of Basic and Clin. Pharmacy, V. 7, Issue 2 March-May (2017)

The interior space of the structures of the inner ear is limited. The volume of pharmaceutical composition administered directly into the inner ear structures should be carefully monitored, as forcing too much composition will damage the sensory epithelium. For a human patient, the volume administered is preferably about 10 μl to about 2 ml (e.g., from about 25 μl to about 1.5 ml) of composition. For example, from about 50 μl to about 1 ml (e.g., about 100 μl, 200 μl, 300 μl, 400 μl, 500 μl, 600 μl, 700 μl, 800 μl or 900 μl) of composition can be administered. In one embodiment, the entire fluid contents of the inner ear structure, e.g., the cochlea or semi-circular canals, is replaced with pharmaceutical composition. In another embodiment, a pharmaceutical composition of the invention is slowly released into the inner ear structure, such that mechanical trauma is minimized.

It can be advantageous to administer two or more (i.e., multiple) doses of the drug harboring an inhibitor of EGFR signaling. The inventive methods provide for administration of multiple doses of a drug to change the sensory perception of a subject. In some embodiments, at least two doses can be administered to the same ear. Also preferably, the sensory epithelium of the inner ear is contacted with two doses or more of the drug within about 30 days. Preferably, two or more applications are administered to the inner ear within about 90 days. However, three, four, five, six, or more doses can be administered in any time frame (e.g., 2, 7, 10, 14, 21, 28, 35, 42, 49, 56, 63, 70, 77, 85 or more days between doses).

In some embodiments, a drug is administered at a dose from a range of 0.1 to 100 mg/kg. In some embodiments, a drug is administered from a range of 0.1 to 1.0 mg/kg, from 1.0 to 10.0 mg/kg, from 10.0 to 20.0 mg/mg, from 20.0 to 30.0 mg/kg, from 30.0 to 40.0 mg/kg, from 40.0 to 50.0 mg/kg, from 50.0 to 60.0 mg/kg, from 60.0 to 70.0 mg/kg, from 70.0 to 80.0 mg/kg, from 80.0 to 90.0 mg/kg, from 90.0 to 100.0 mg/kg, or any value between the aforementioned values. In some embodiments, a drug is administered at a dose of 10 mg/kg, p.o., q.d. (for 14 days). In some embodiments, a drug is administered at a first dose each day for a selected period of time, and then at a second dose each day for a selected period of time. In some embodiments, the first and second doses are independently selected from 0.6 mg/kg, 5 mg/kg, 10 mg/kg, 30 mg/kg, 60/mg/kg. In some embodiments, the first and second doses are independently selected from 0.5 to 30 mg/kg, or any dose between the aforementioned values. The selected period of time is from 1 to 30 days, from 1 to 14 days, or each day throughout the subject's life following the first administration of the drug. All descriptions with respect to dosing, unless otherwise expressly stated, apply to the compounds of the invention, including cytoprotective drugs of this disclosure.

Pharmaceutical Composition. In some embodiments, the compounds of this disclosure can be a drug. A drug of the invention desirably is administered in a pharmaceutical composition, which includes a pharmaceutically acceptable carrier and the drug. Any suitable pharmaceutically acceptable carrier can be used within the context of the invention, and such carriers are well known in the art. The choice of carrier will be determined, in part, by the particular site to which the composition is to be administered and the particular method used to administer the composition.

Suitable formulations include aqueous and non-aqueous solutions, isotonic sterile solutions, which can contain anti-oxidants, buffers, bacteriostats, and solutes that render the formulation isotonic with the blood or fluid of the inner ear of the intended recipient, and aqueous and non-aqueous sterile suspensions that can include suspending agents, solubilizers, thickening agents, stabilizers, and preservatives. The formulation can include artificial endolymph or perilymph, which are commercially available. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Preferably, the pharmaceutically acceptable carrier is a buffered saline solution. In some embodiments, the pharmaceutical compositions of this disclosure includes a pharmaceutically acceptable liquid carrier. Such liquid carriers can include or exclude: polysorbate 80, L-arginine, polyvinylpyrrolidone, trehalose, and combinations thereof.

The method of the invention can be part of a treatment regimen involving other therapeutic modalities. It is appropriate, therefore, if the inventive method is employed to prophylactically or therapeutically treat a sensory disorder, namely a hearing disorder or a balance disorder, that has been treated, is being treated, or will be treated with any of a number of other therapies, such as drug therapy or surgery. The inventive method also can be performed in conjunction with the implantation of hearing devices, such as cochlear implants. The inventive method also is particularly suited for procedures involving stem cells to regenerate populations of cells within the inner ear.

The inventive method also can involve the co-administration of other pharmaceutically active compounds. By “co-administration” is meant administration before, concurrently with, e.g., in combination with another pharmaceutically active compound in the same formulation or in separate formulations, or after administration of the other pharmaceutically active compound as described above. For example, factors that control inflammation, such as ibuprofen or steroids, can be co-administered to reduce swelling and inflammation associated with administration of the other pharmaceutically active compound. Immunosuppressive agents can be co-administered to reduce inappropriate immune responses related to an inner ear disorder or the practice of the inventive method. Similarly, vitamins and minerals, antioxidants, and micronutrients can be co-administered. Antibiotics, i.e., microbicides and fungicides, can be co-administered to reduce the risk of infection associated with surgical procedures.

### Kits

The present disclosure also provides any of the above-mentioned compositions in kits or commercial packages, optionally with instructions for use or administration of any of the compositions described herein by any suitable technique as previously described. “Instructions” can define a component of promotion, and typically involve written instructions on or associated with packaging of compositions of the invention. Instructions also can include any oral or electronic instructions provided in any manner. The “kit” typically defines a package including any one or a combination of the compositions of the invention and the instructions but can also include the composition of the invention and instructions of any form that are provided in connection with the composition in a manner such that a clinical professional will clearly recognize that the instructions are to be associated with the specific composition.

The kits described herein may also contain one or more containers, which may contain the inventive inhalable formulation and other ingredients as previously described. The kits also may contain instructions for mixing, diluting, and/or administrating the compositions of the invention in some cases. The kits also can include other containers with one or more solvents, surfactants, preservative and/or diluents (e.g., normal saline (0.9% NaCl)) and containers for mixing, diluting or administering the components in a sample or to a subject in need of such treatment.

The compositions of the kit may be provided as any suitable form, for example, as liquid solutions or as dried powders. When the composition provided is a dry powder, the composition may be reconstituted by the addition of a suitable diluent, which may also be provided. In embodiments where liquid forms of the composition are used, the liquid form may be concentrated or ready to use. The diluent will depend on the components of the composition and the mode of use or administration. The diluent will depend on the conjugate and the mode of use or administration.

### EXAMPLES

The following non-limiting examples are provided to further illustrate the present invention.

### Materials and Methods

### Mouse Model

For the single-dose cisplatin protocol, FVB/NJ breeding mice were purchased from Jackson Laboratory (Bar Harbor, Maine, USA), bred in the animal facility at Creighton University, and used at 6-8 weeks old for the single dose cisplatin experiment. For the multi-cycle cisplatin protocol, 8-week-old CBA/CaJ mice were purchased from Jackson Laboratory with an equal number of males and females. The CBA/CaJ mice were given one week to acclimate to the Animal Resource Facilities at Creighton University. Animals were anesthetized by Avertin (2,2,2-tribromoethanol) via intraperitoneal injection at a dose of 500 mg/kg, and complete anesthetization was determined via toe pinch. For all experiments, mice were randomly assigned to experimental groups, maintaining a balance of males and females in each group.

### Single Dose Cisplatin Treatment in Mice

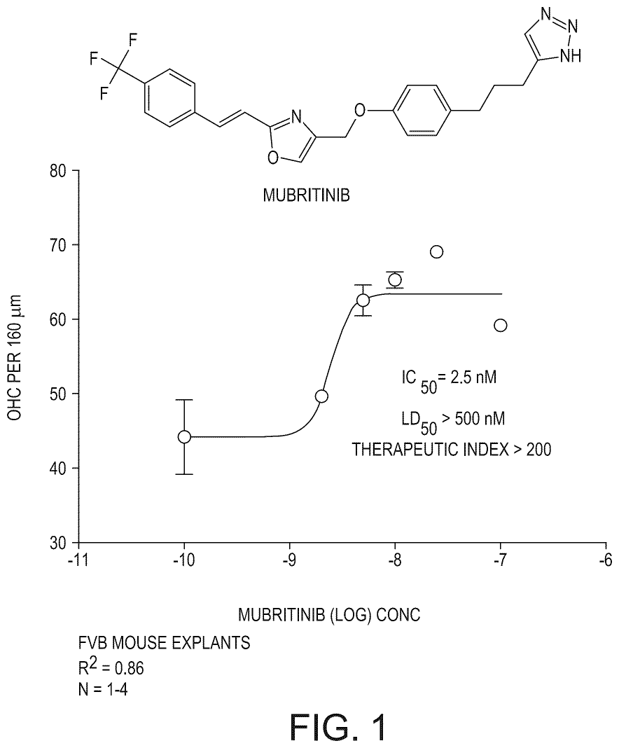

10 milligrams of cisplatin (479306, Sigma-Aldrich) powder was dissolved in 10 mL of sterile saline (0.9% NaCl) at 37° C. for 40 to 60 minutes. 30 mg/kg was administered once to FVB mice via intraperitoneal injection on day 1 of the protocol (FIG. 1A) (6,20). One day before cisplatin injection, mice received 1 mL of saline by subcutaneous injection and were given 1 mL of saline twice a day throughout the protocol until body weight started to recover. The cages of cisplatin treated mice were placed on heating pads until body weights began to recover. Food pellets dipped in DIETGEL Boost (ClearH20 Westbrook, Maine were placed on the cage floor of cisplatin-treated mice. DIETGEL Boost (ClearH20 Westbrook, Maine is a high calorie dietary supplement that provides extra calorie support for mice. The investigators and veterinary staff carefully monitored for changes in overall health and activity that may have resulted from cisplatin treatment.

### Multi-Cycle Cisplatin Treatment in Mice

4.5 milligrams of cisplatin (479306, Sigma-Aldrich) powder was dissolved in 25 mL of sterile saline (0.9% NaCl) at 37° C. for 40 to 60 minutes. 3 mg/kg cisplatin was administered to mice via intraperitoneal injection once a day in the morning. This repeated for 4 total days with a 10-day recover period in which no cisplatin was administered to the mice. Mice were treated with 3 mg/kg cisplatin for a total of 12 days (4 days per cycle with 3 cycles) (FIG. 2A) (46,47). Cisplatin treated mice were injected by subcutaneous injection twice a day with 1 mL of warm saline to ameliorate dehydration. This continued until body weight started to recover. The cages of cisplatin-treated mice were placed on heating pads throughout the duration of the experiment until mice began to recover after the 3rd treatment cycle of the protocol. Food pellets dipped in DIETGEL Boost (ClearH20 Westbrook, Maine). were placed on the cage floor of cisplatin-treated mice. The investigators and veterinary staff carefully monitored for changes in overall health and activity that may have resulted from cisplatin treatment.

### Compound Administration by Oral Gavage

The compound dabrafenib mesylate was purchased from MedChemExpress and administered to FVB/NJ and CBA/CaJ mice via oral gavage. Dabrafenib was dissolved in a mixture of 10% DMSO, 5% Tween 80, 40% PEG-E-300, and 45% saline. For the single dose cisplatin experiment, 12 mg/kg dabrafenib was given to mice once in the morning and once at night. This continued for a total of 3 days (FIG. 1A). For the multi-cycle cisplatin protocol, 15, 3, or 0.6 mg/kg dabrafenib was administered once in the morning and once at night for 4 total days with a 10-day recovery period in which no dabrafenib was administered to the mice. This cycle was repeated a total of 3 times (FIG. 2A). Mice treated with cisplatin and dabrafenib were given dabrafenib 1 hour before treatment with cisplatin in the morning.

ABR threshold and wave 1 amplitude measurements

ABR waveforms in anesthetized mice were recorded in a sound booth by using subdermal needles positioned in the skull, below the pinna, and at the base of the tail, and the responses were fed into a low-impedance Medusa digital biological amplifier system (RA4L; TDT; 20-dB gain). At the tested frequencies (8, 16, and 32 kHz), the stimulus intensity was reduced in 10-dB steps from 90 to 10 dB to determine the hearing threshold. ABR waveforms were averaged in response to 500 tone bursts with the recorded signals filtered by a band-pass filter from 300 Hz to 3 kHz. ABR threshold was determined by the presence of at least 3 of the 5 waveform peaks (6,20). Baseline ABR recordings before any treatment were performed when mice were 6-7 weeks old for the single dose cisplatin experiments and 9 weeks old for the multi-dose cisplatin protocol. All beginning threshold values were between 10 and 40 dB at all tested frequencies. In the single dose cisplatin experiment, post-treatment recordings were performed 21 days following cisplatin treatment. For the multi-cycle cisplatin protocol, post-treatment recordings were performed 42 days after the start of the 3-cycle protocol (aged 18 weeks) with half the mice kept alive and ABR was performed again on these mice 4 months after the completion of the 42-day treatment protocol. All thresholds were determined independently by two-three experimenters for each mouse who were blind to the treatment the mice received. ABR wave one amplitudes were measured as the difference between the peak of wave 1 and the noise floor of the ABR trace.

### DPOAE Measurements

Distortion product otoacoustic emissions were recorded in a sound booth while mice were anesthetized. DPOAE measurements were recorded using the TDT RZ6 processor and BioSigTZ software. The ER10B+ microphone system was inserted into the ear canal in way that allowed for the path to the tympanic membrane to be unobstructed. DPOAE measurements occurred at 8, 16, and 32 kHz with an f2/f1 ratio of 1.2. Tone 1 was *0.909 of the center frequency and tone 2 was *1.09 of the center frequency. DPOAE data was recorded every 20.97 milliseconds and average 513 times at each intensity level and frequency. At each tested frequency, the stimulus intensity was reduced in 10 dB steps starting at 90 dB and ending at 10 dB. DPOAE threshold was determined by the presence of an emission above the noise floor. Baseline DPOAE recordings occurred when CBA/CaJ mice were 10 weeks old and tested again on day 42 (immediately after cycle 3) and on day 165 (4 months after cycle 3). DPOAE threshold shifts were determined by subtracting the baseline DPOAE recording from the post experimental recording.

### Tissue Preparation, Immunofluorescence, and OHCs Counts.

Cochleae from adult mice were prepared and examined as described previously (80-82). Cochleae samples were immunostained with anti-myosin VI (1:400; 25-6791, Proteus Bioscience) or pERK antibody (1:400; 9101 L, Cell Signaling) with secondary antibodies purchased from Invitrogen coupled to anti-rabbit Alexa Fluor 488 (1:400; A11034). All images were acquired with a confocal microscope (LSM 700 or 710, Zeiss). Outer hair cell counts were determined by the total amount of outer hair cells in a 160 μm region (6,20,82). Counts were determined for the 8, 16, and 32 kHz regions. Cochleae from each experimental group were randomly selected to be imaged for outer hair cell counts.

### Endocochlear Potential Measurements

Mice were anesthetized using a combined regimen of ketamine (16.6 mg/ml) and xylazine (2.3 mg/ml) and supplemented as needed to maintain a surgical level via intraperitoneal injection. For recording the EP, a round-window approach was used. A glass capillary pipette electrode (10 MU) was mounted on a hydraulic micromanipulator and advanced until a stable positive potential was observed. Signals were filtered and amplified under current-clamp mode using an Axopatch 200B amplifier (Molecular Devices, San Jose, CA) and acquired by software pClamp 9.2. The sampling frequency was 10 kHz.

### Kidney Histology Examination

Following cisplatin and dabrafenib treatment, mice were sacrificed, and kidneys were extracted and put into 4% PFA. The kidneys were later embedded in paraffin, sectioned (3 μm), and stained with Hematoxylin and Eosin (H&E) and Periodic Acid-Schiff (PAS). Sections were observed under a microscope (Nikon Eclipse Ci) for histological examination. A semi-quantitative pathological scoring system was used as described in Pabla et al, 2015 and Hu et al, 2010 (54,55). The grading system uses scores 0-4 that indicate the percentage of damage in each section. Sections were analyzed by an experienced pathologist in a double-blind manner. The grades are: grade 0 (minimal)=<10% damage with no visible lesions and normal morphology; grade 1 (mild)=11-25% damage with mild tubule dilation, swelling of cells, presence of luminal debris or cast and nuclear condensation with partial loss of brush borders in ⅓ tubules; grade 2 (moderate)=26-50% damage with clear dilation of tubules, loss of brush borders, nuclear loss and presence of casts in <⅔ of tubules; Grade 3 (marked)=51-75% damage with severe dilation of most tubule, total loss of brush borders and nuclear loss in ⅔ tubule and grade 4 (severe)=>75% damage with complete loss of tissue morphology, severe tubule dilation and loss of nucleus and brush borders.

### Liver Histology Examination

Following cisplatin and dabrafenib treatment, mice were sacrificed, and livers were extracted and put into 4% PFA. The livers were later embedded in paraffin, sectioned (3 μm), and stained with Hematoxylin and Eosin (H&E) and Masson's trichrome stain. Sections were observed under a microscope (Nikon Eclipse Ci) for histological examination. The grading system uses a score of 0-4 that indicates the amount of damage in each section. Sections were analyzed by an experienced pathologist in a double-blind manner. The grades are grade 0 (normal), grade 1 (mild damage), grade 2 (moderate damage), grade 3 (severe damage), and grade 4 (very severe/fulminant damage). Criteria that determined the scoring of each liver sample was the presence of fibrosis, lobular disarray, hepatocyte swelling, hepatocyte nuclear changes, hepatocyte necrosis, lobular inflammation, portal inflammation, sinusoidal and central vein congestion, and Kupffer cell hyperplasia.

### Statistical Analysis

Statistics was performed using Prism (GraphPad Software). Two-way analysis of variance (ANOVA) with Bonferroni post hoc test was used to determine mean difference and statistical significance. Statistical significance was determined when P<0.05.

### Example 1: EGFR Inhibitors for Protection Against Hearing Loss

A screen of a library composed of 75 kinase inhibitors was conducted to identify inhibitors that protect against cisplatin-induced hair cell loss. This screen identified four compounds: (1) Her2 inhibitor MUBRITINIB (TAK 165), (2) Pan-AUR inhibitor SNS314 (3) BRAF-V600E inhibitor GSK2118436A (DABRAFENIB), and (4) PDGFR inhibitor CRENOLANIB that potently protected against cisplatin-induced cell death in a mouse cochlea-derived cell line (HEI-OC1) and cisplatin-induced hair cell loss in mouse cochlear explants.

Her2 inhibitor MUBRITINIB (TAK 165) exhibited protective effects against cisplatin-induced Caspase-3/7 activity in HEI-OC1 cells with an IC₅₀ of 4 nM and LD₅₀ of >55 μM; and protected against cisplatin-induced hair cell loss in mouse cochlear explants with IC₅₀ of 2.5 nM and LD₅₀ of >500 nM (Therapeutic Index of >200) (FIG. 1). Similarly, the pan-ErbB inhibitor, PELITINIB, was found to exhibit protective effects against cisplatin-induced Caspase-3/7 activity in HEI-OC1 cell loss with IC₅₀ of 0.6 μM and LD₅₀ of 40 μM (FIG. 2). Moreover, with 1 hour pre-incubation, PELITINIB exhibited 49% protection of outer hair cells against cisplatin-induced hair cell loss in mouse cochlear explants (N=3).

Similarly, the pan-ErbB inhibitor, PELITINIB, was found to exhibit protective effects against cisplatin-induced Caspase-3/7 activity in HEI-OC1 cell loss with IC₅₀ of 0.6 μM and LD₅₀ of 40 μM (FIG. 2). Moreover, with 1 hour pre-incubation, PELITINIB exhibited 49% protection of outer hair cells against cisplatin-induced hair cell loss in mouse cochlear explants (N=3).

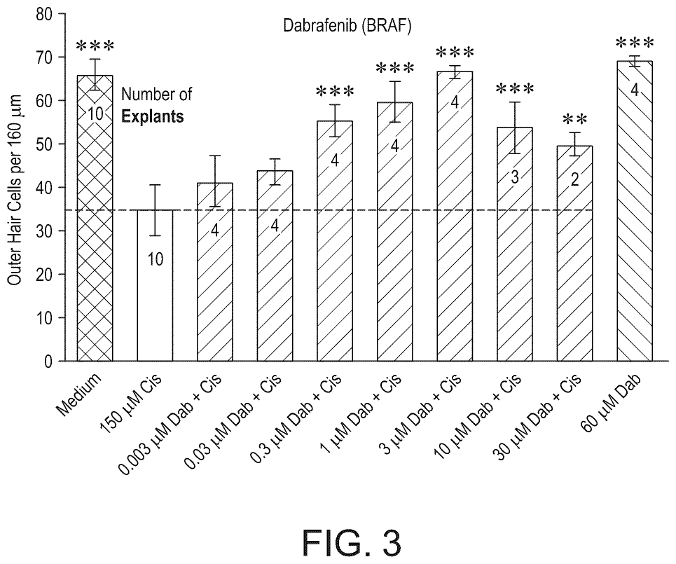

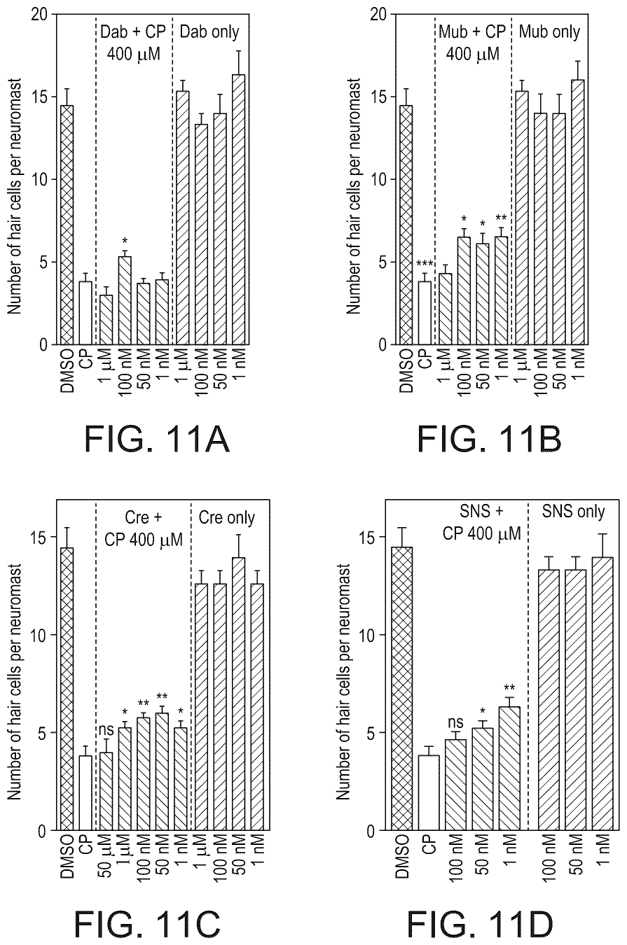

B-Raf inhibitors protected outer hair cells against cisplatin injury in mouse cochlear explants. B-Raf inhibitor Dabrafenib (BRAF) (FIG. 3A) exhibited protective cisplatin-induced hair cell loss in mouse cochlear explants with IC₅₀ of 0.0300 μM and LD₅₀ of 13.47 μM (Therapeutic Index of greater than 2000). Additionally, Dabrafenib (BRAF) showed Zebrafish compound cisplatin protection at 0.100 μM (FIG. 11A).

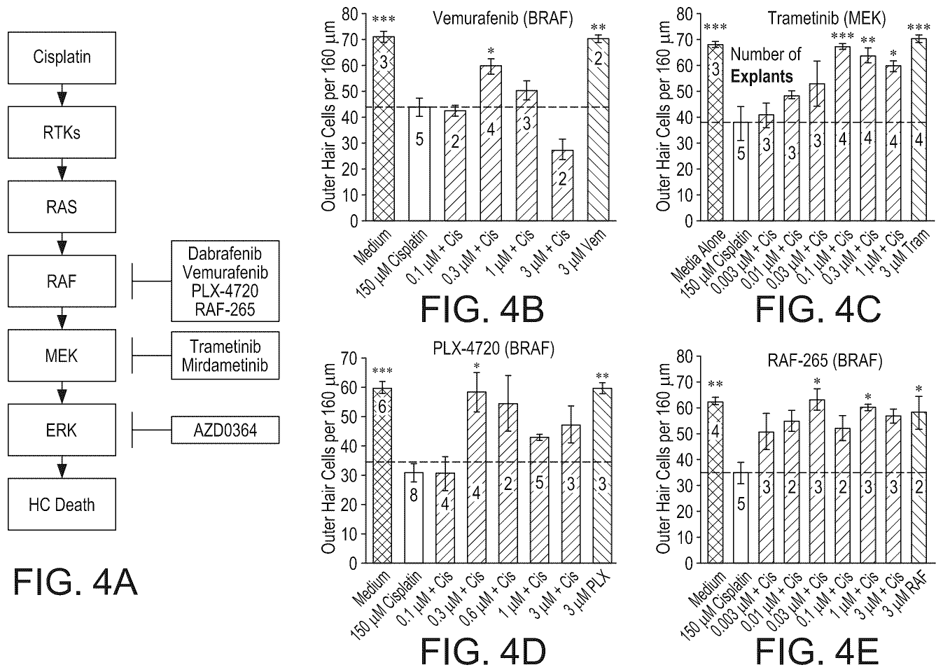

Additionally, the B-Raf inhibitor VEMURAFENIB (BRAF) (FIG. 4B) exhibited protective cisplatin-induced hair cell loss in mouse cochlear explants with IC₅₀ of ˜0.2 μM and LD₅₀ of greater than 3 μM (Therapeutic Index of greater than 15). B-Raf inhibitor PLX-4750 (BRAF) (FIG. 4D) exhibited protective cisplatin-induced hair cell loss in mouse cochlear explants with IC₅₀ of 0.2 μM and LD₅₀ of greater than 3 μM. (Therapeutic Index of greater than 15). B-Raf inhibitor RAF-265 (BRAF) (FIG. 4E) exhibited protective cisplatin-induced hair cell loss in mouse cochlear explants with IC₅₀ of 0.02 μM and LD₅₀ of greater than 3 μM (Therapeutic Index of greater than 150).

MEK inhibitors protected outer hair cells against cisplatin injury in cochlear explants as shown in FIG. 4C MEK inhibitor TRAMETINIB (MEK) exhibited protective cisplatin-induced hair cell loss in mouse cochlear explants with IC₅₀ of 0.05 μM and LD₅₀ of greater than 3 μM (Therapeutic Index of greater than 60).

As illustrated in FIG. 4A inhibitor mitigate cisplatin along the signaling cascade.

### Example 2 Inhibitors Mitigate Cisplatin Activated B-Raf Signaling Cascade

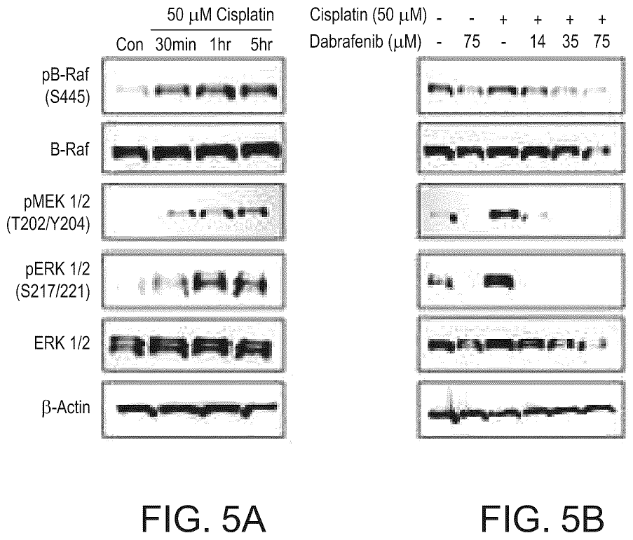

As shown in FIGS. 5A and 5B, B-Raf, ERK, and MEK become phosphorylated and activated in HEL-OC1 cells upon 50 μM cisplatin treatment in a time-dependent manner. Dabrafenib treatment mitigates cisplatin-mediated 50 PM, 1 hour activation of B-RAF, ERK and MEK in HEL-OC1 cells in a does dependent manner.

### Example 3. DABRAFENIB is Protective Against Cisplatin-Induced Hearing Loss in Adult Mice when Delivered Orally In Vivo

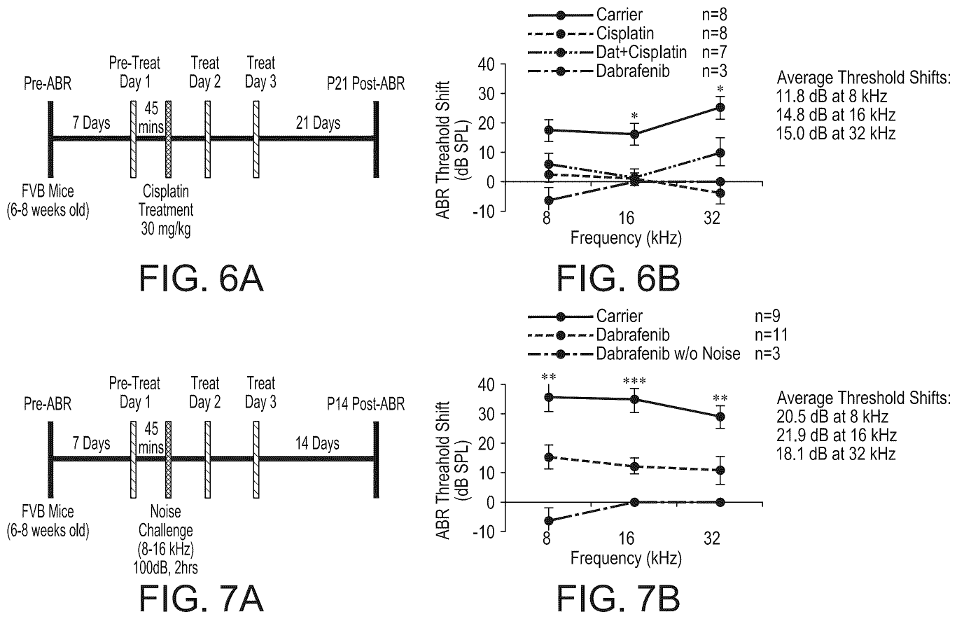

FIG. 6A shows a schedule of administration of dabrafenib and cisplatin to adult FVB mice (males and females). FIG. 6B shows reduced ABR threshold shifts of 11.8-15.0 dB on average were recorded on day 21 after first day of cisplatin and dabrafenib co-treatment, mean±SEM, *, P<0.05, compared to cisplatin alone by two-way ANOVA followed by a Bonferroni comparison.

### Example 4 DABRAFENIB is Protective Against Noise-Induced Hearing Loss in Adult Mice when Delivered Orally In Vivo

FIG. 7A shows the schedule of administration of dabrafenib and noise exposure to FVB mice (males and females). FIG. 7B shows reduced ABR threshold shifts of 18.1-21.9 dB on average were recorded on day 14 after first day of dabrafenib and noise exposure, mean±SEM, **, P<0.01, ***, P<0.001, compared to carrier by two-way ANOVA followed by a Bonferroni comparison.

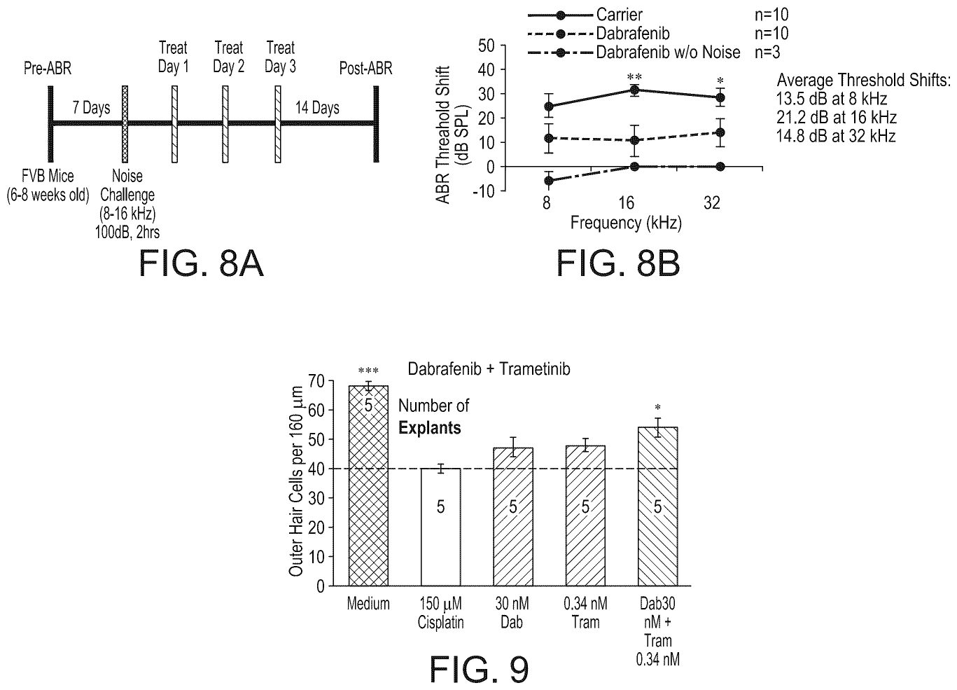

Now referring to FIGS. 8A and 8B, Dabrafenib is protective against noise-induced hearing loss in adult mice when delivered orally forty-five minutes before the noise exposure. (A) Schedule of administration of dabrafenib and noise exposure to FVB mice (males and females). (B) Reduced ABR threshold shifts of 18.1-21.9 dB in average were recorded on day 14 after first day of dabrafenib and noise exposure, mean±SEM, **, P<0.01, ***, P<0.001, compared to carrier by two-way ANOVA followed by a Bonferroni comparison.

### Example 5: A Combination of Inhibitors Act Synergistically

Now referring to FIG. 9, testing of a B-Raf/MEK1/2 inhibitor combination in mouse cochlear explant cultures. Compounds alone or combination of the compounds were added 1 h before cisplatin (150 μM) to P3 FVB cochlear explants for 24 h, and number of outer hair cells per 160 μm of middle turn regions of the cochlea were counted by phalloidin staining, mean±SEM, P=*<0.05, P=***<0.001, compared to cisplatin alone by unpaired two-tailed Student's t-test. The initial molar ratio between the compounds tested was determined by the ratio given currently to cancer patients (dabrafenib at 150 mg twice daily plus trametinib at 2 mg once daily). It should be noted that the synergistic treatment is evidenced by both the number of outer hair cell count, and the otoprotective or otorestorative evidence as described in these examples.

### Example 6 Protective Effects are Shown in Zebrafish Lateral Line Neuromasts In Vivo

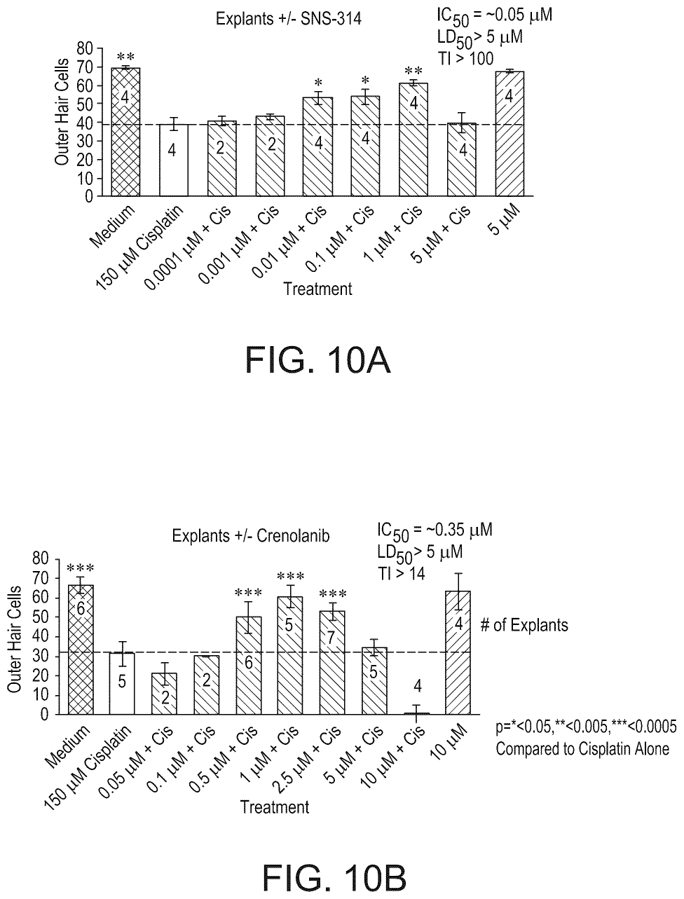

Methods of using a zebrafish model system to evaluate small molecules capable of decreasing, inhibiting or preventing sensory hair cell damage or death are provided. Zebrafish are an advantageous animal model system for studying causes and prevention of hearing loss in comparison to mammalian animal model systems. The relative inaccessibility of hair cells in mammalian organisms limits their use as a high throughput model for identifying compounds that would prevent toxin mediated and other forms of hair cell death from occurring. The lateral line neuromast hair cells of zebrafish (Danio rerio) are structurally and functionally similar to mammalian sensory hair cells. Compounds SNS-314 (FIG. 10A) and crenolanib (FIG. 10B) protected in the cochlear explant culture assay against cisplatin-induced hair cell death. Dose-response of compounds SNS-314 and crenolanib in mouse cochlear explants treated with or without cisplatin. Compounds alone or compounds added 1 h before cisplatin (150 μM) to P3 FVB cochlear explants for 24 h, and number of outer hair cells per 160 μm of middle turn regions of the cochlea were counted by phalloidin staining, mean±SEM, P=*<0.05, P=**<0.005, ***P<0.0005 compared to cisplatin alone by unpaired two-tailed Student's t-test.

Protection of zebrafish lateral line neuromast hair cells in vivo. Five days post-fertilization Tg(brn3c: GFP) larvae were incubated with vehicle alone (DMSO), 400 μM of cisplatin (CP) for 6 hours or pre-treated with one of the compounds: dabrafenib (FIG. 11A), mubritinib (FIG. 11B), crenolanib (FIG. 11C) or SNS-314 (FIG. 11D) for 1 hour followed by a 6 hours co-incubation with the compound tested and CP 400 μM. After the treatment, animals were transferred to fresh fish water to recover for 1 hour and then fixed and immunostained for GFP and otoferlin. Quantification of the number of hair cells per neuromast after the different treatments represented as mean+/−SEM. Student's t test was performed, *p<0.05, **p<0.01, ***p<0.001, compared versus CP 400 μM. Neuromasts inspected: O03 (supraorbital line neuromast) and O1-2 (otic line neuromasts) from at least 3 different animals.

### Example 7 Synergistic Effect of Two Inhibitors

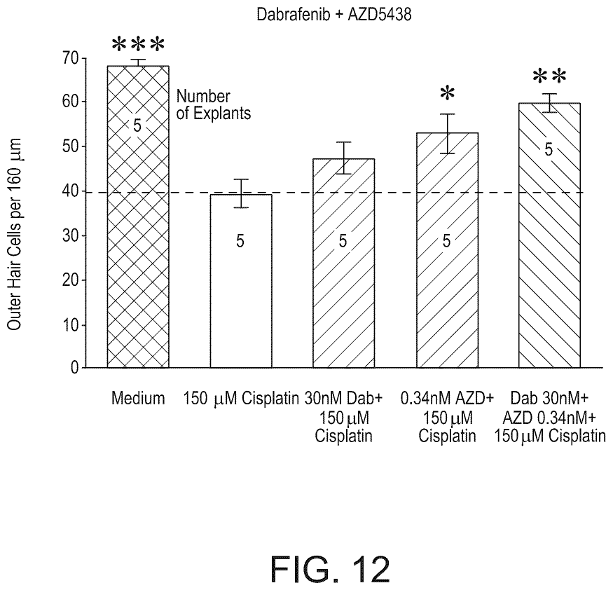

FIG. 12 shows the compounds Dabrafenib (a B-Raf kinase inhibitor) and AZD5438 (a CDK2 kinase inhibitor) protects against cisplatin and noise ototoxicity, better than individual inhibitor, in mouse cochlear explants and mice in vivo. B-Raf/CDK2 inhibitor combination protects cisplatin induced hair cell loss in mouse cochlear explants. Compounds alone (purple bar) or combination of the compounds were added 1 h before cisplatin (150 μM) to P3 FVB cochlear explants for 24 hrs, and number of outer hair cells per 160 μm of middle turn regions of the cochlea were counted by phalloidin staining, mean±SEM, P=*<0.05, P=**<0.005, compared to cisplatin alone by unpaired two-tailed Student's t-test. The initial AZD5438/dabrafenib combination tested was in the same molar ratio (0.34/30).

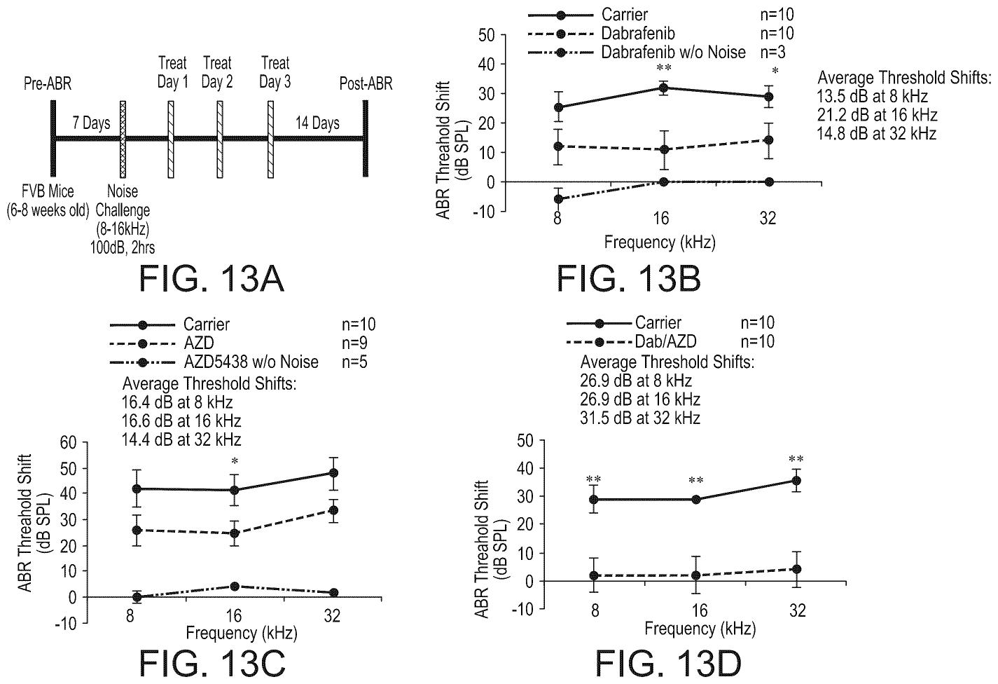

FIG. 13A-13D show oral delivery of the combination of inhibitors provides protection effects that are significantly better than the use of any individual compound alone.

B-Raf/CDK2 inhibitor combination protects fully against noise induced hearing loss in mice when delivered orally. In this example, compounds for oral delivery were dissolved in the carrier 10% DMSO, 40% PEG300, 5% Tween-80 and 45% saline (0.9% NaCl) and were given in a volume of 10 ml/kg. Schedules of drugs and noise levels are shown in FIG. 13A. In FIG. 13B Dabrafenib (2×60 mg/kg/day) is given by oral delivery continuously for 3 days post-noise. In FIG. 13C AZD5438 (2×35 mg/kg/day) is given by oral delivery continuously for 3 days post-noise. In FIG. 13D complete protection against noise is achieved with combination of the two drugs for 3 days post-noise (dabrafenib (2×60 mg/kg/day) and AZD5438 (2×35 mg/kg/day), mean±SEM, *, P<0.05, **, P<0.01, compared to carrier alone by unpaired two-tailed Student's t-test

### Example 7. Dabrafenib as Representative EGFR Inhibitor Restores Hearing Loss while Maintaining Subject Body Weight

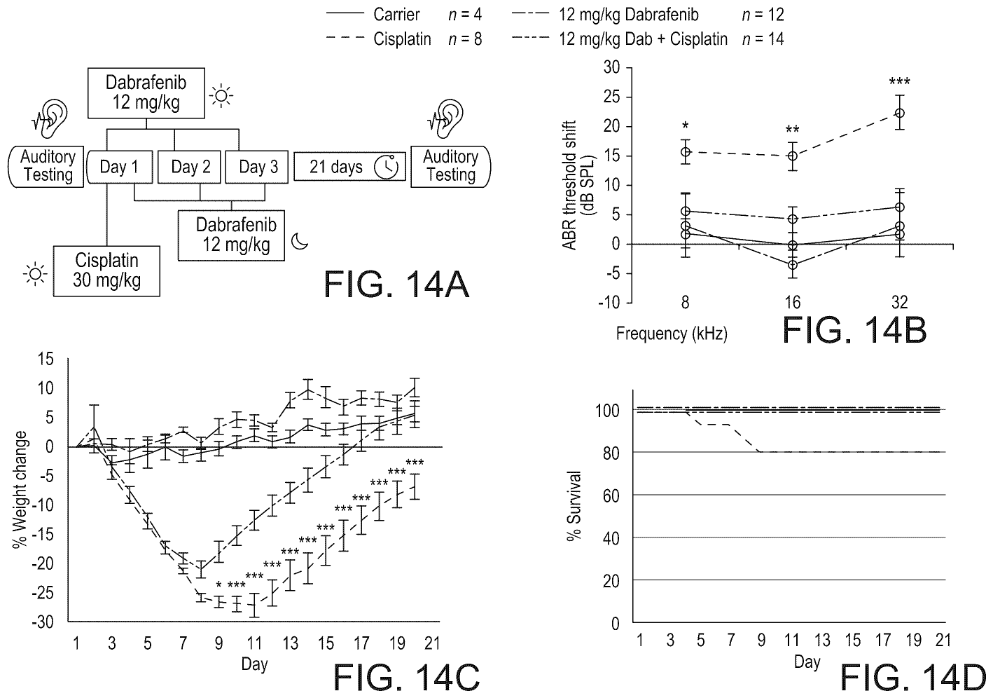

A lower dose of 12 mg/kg dabrafenib using the single-dose cisplatin protocol to compare it to the previously used 100 mg/kg dabrafenib dose. Dabrafenib was administered twice daily by oral gavage, for three consecutive days, with the first dose given 45 minutes before cisplatin injection (FIG. 14A). Dabrafenib provided significant protection from cisplatin-induced hearing loss by ABR functional hearing measurements at 8, 16, and 32 kHz frequencies, with the greatest protection observed at 32 kHz. The average protection achieved was 10 dB SPL at 8 kHz, 10 dB at 16 kHz, and 16 dB at 32 kHz. Twice daily 12 mg/kg dabrafenib (40% of the human equivalent dose) provided equivalent hearing protection to previously tested once-daily 100 mg/kg dose (FIG. 14B). Mice administered both dabrafenib and cisplatin experienced a significant reduction in weight loss, beginning on day 9 and persisting through day 21, compared to the cisplatin alone treated cohort, while those treated with dabrafenib alone exhibited no change in weight compared to carrier alone (FIG. 14C). Additionally, no mouse death occurred in cohorts treated with dabrafenib and cisplatin, while 20% of mice treated with cisplatin alone died (FIG. 14D).

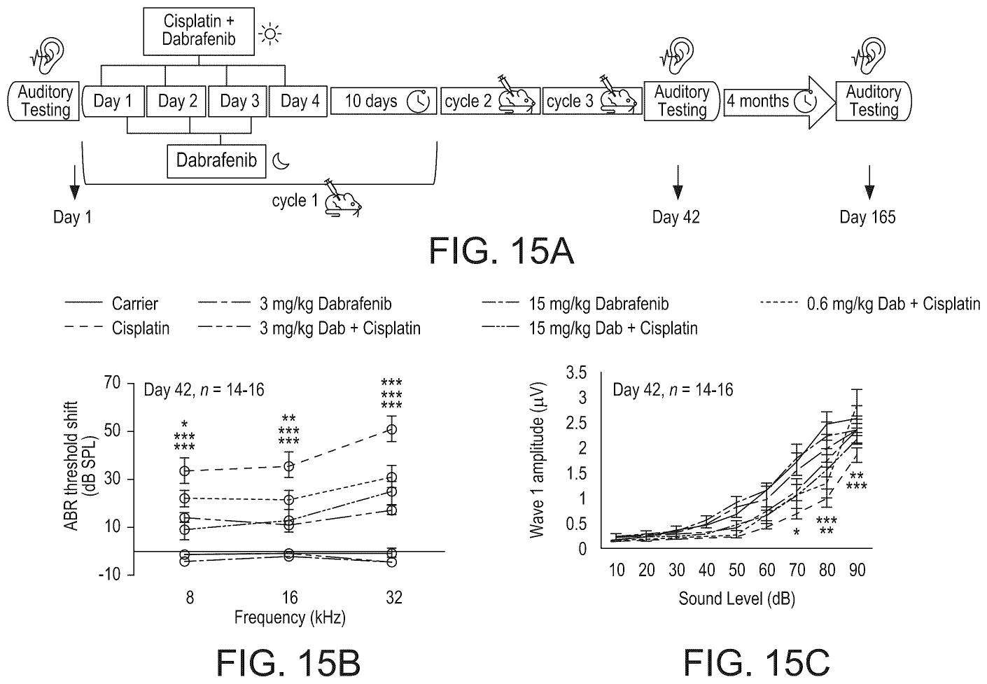

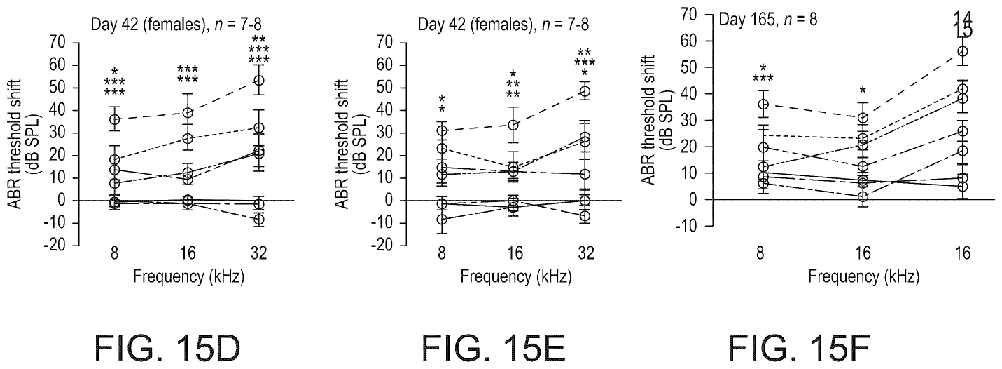

Now referring to FIGS. 15A-15F it can be observed that dabrafenib protects against cisplatin-induced hearing loss using a multi-cycle, low-dose cisplatin treatment regimen. The term multi-cycle means administration of the drug means repeated treatment over a set period of time as exemplified in FIG. 15A. Human subjects treated with cisplatin are not administered a single, high dose. In this example, the efficacy of dabrafenib to protect from cisplatin ototoxicity in a clinically relevant mouse model following a protocol initially developed by Roy et al. 2013 and optimized by Fernandez at al. An optimized, clinically relevant mouse model of cisplatin-induced ototoxicity. Hearing research. 2019; 375:66-74. The multi-cycle treatment regimen includes a first cycle wherein the drugs are administered for a few days (exemplary number of 4 days) and then a rest period of 10 days. The second cycle commence after the 10 day rest period and includes the steps of wherein the drugs are administered for a few days (exemplary number of 4 days) and then a rest period of 10 days. The third cycle commence after the 10 day rest period and includes the steps of wherein the drugs are administered for a few days (exemplary number of 4 days) and then a rest period of 10 days. After the ten day rest period the auditory testing takes place this is at 42 days post the initial treatment. The protective effect has been observed for 4 months.

The second cycle commence after the 10 day rest period and includes the steps of wherein the drugs are administered for a few days (exemplary number of 4 days) and then a rest period of 10 days.

The doses of dabrafenib tested in this study are 15, 3, and 0.6 mg/kg. 15 mg/kg was chosen as it is close to the lowest effective dose tested of dabrafenib in the high single-dose cisplatin protocol (12 mg/kg FIG. 14B-C) and two additional 1:5 deescalating doses (3 and 0.6 mg/kg) were selected to determine the drug's minimum effective dose. Dabrafenib at doses of 15 or 3 mg/kg/bw provide significant protection from cisplatin-induced hearing loss in this clinically relevant mouse model. As shown in FIG. 15B, mice co-treated with 15, 3, and 0.6 mg/kg dabrafenib and cisplatin had significantly lower ABR threshold shifts compared to cisplatin alone treated mice with an ABR average threshold shift reduction at 32 kHz of 27, 34, and 20 dB, respectively.

Mice treated with 3 mg/kg dabrafenib had significantly higher ABR wave 1 amplitudes at 16 kHz compared to cisplatin alone at 90-, 80-, and 70-dB SPL, while 15 mg/kg had significantly higher wave 1 amplitude at 80 dB SPL, and 0.6 mg/kg at 90 dB SPL (FIG. 15C). Additionally, mice co-treated with 15 or 3 mg/kg dabrafenib and cisplatin had lower ABR threshold shifts for both males and females at the 8, 16, and 32 kHz frequency regions. Male mice treated with 0.6 mg/kg had significantly lower threshold shifts at 8 and 32 kHz and females at 16 and 32 kHz. (FIGS. 15D & E). Furthermore, the hearing protection of mice given 15 or 3 mg/kg dabrafenib did not diminish 4 months after the completion of the 42-days of treatment, with significant protection maintained at all frequencies tested. The 0.6 mg/kg dabrafenib treated mice lost their protection at this time point (FIG. 15F). No statistically significant difference in ABR threshold shifts was observed between the 15 mg/kg dabrafenib co-treated group and the 3 mg/kg dabrafenib co-treated group,

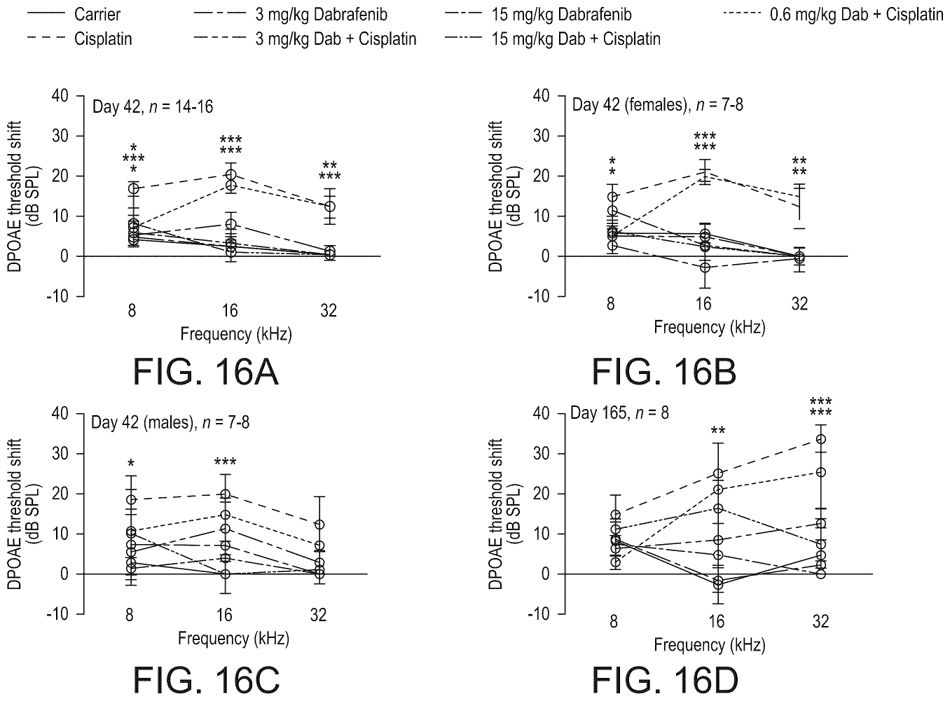

Now referring to FIGS. 16A-16D DPOAE threshold shifts were also calculated immediately after and 4 months following the completion of cycle 3. As shown in FIG. 16A, mice co-treated with 15 or 3 mg/kg dabrafenib and cisplatin had significantly lower DPOAE threshold shifts compared to the cisplatin alone treated mice with a reduction in average DPOAE threshold shifts at 16 kHz of 19- and 13-dB SPL, respectively. Co-treatment of cisplatin and dabrafenib at 0.6 mg/kg had significantly lower DPOAE threshold shift at 8 kHz only immediately after the completion of cycle 3 (FIG. 16A). Males and females were analyzed separately and dabrafenib co-treated mice with cisplatin had significantly lower DPOAE threshold shifts in both sexes (FIG. 16B and FIG. 16C). 3 mg/kg dabrafenib co-treatment with cisplatin had significantly lower DPAOE threshold shifts at all 3 tested frequencies in females, while males had significantly lower threshold shifts at 8 kHz. 15 mg/kg dabrafenib co-treatment with cisplatin had significantly lower DPOAE threshold shifts at the 16 and 32 kHz frequencies in females and 16 kHz in males. 0.6 mg/kg dabrafenib co-treatment with cisplatin had significantly lower DPOAE threshold shifts at 8 kHz in females only. DPOAE threshold shifts measured at 4 months after the completion of cycle 3 show mice co-treated with 3 mg/kg dabrafenib and cisplatin had significantly lower threshold shifts at 16 and 32 kHz, while 15 mg/kg dabrafenib and cisplatin treated mice had significance at 32 kHz (FIG. 16D). The term co-treatment means the drugs are administered at the same time or can be premixed as a formulation.

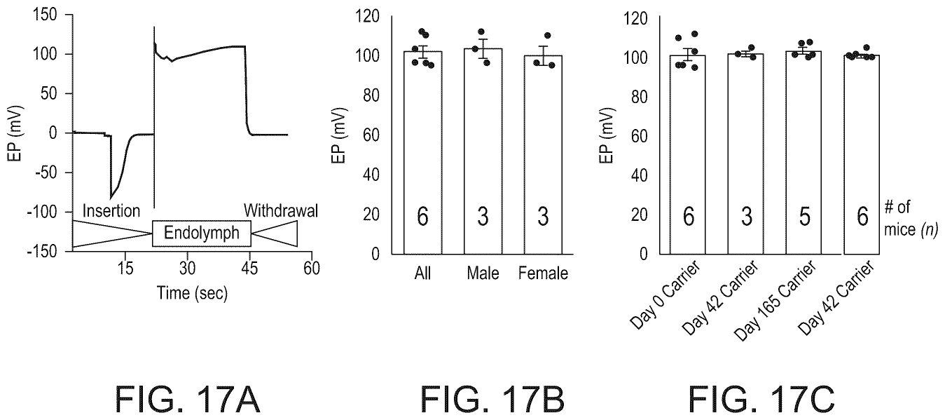

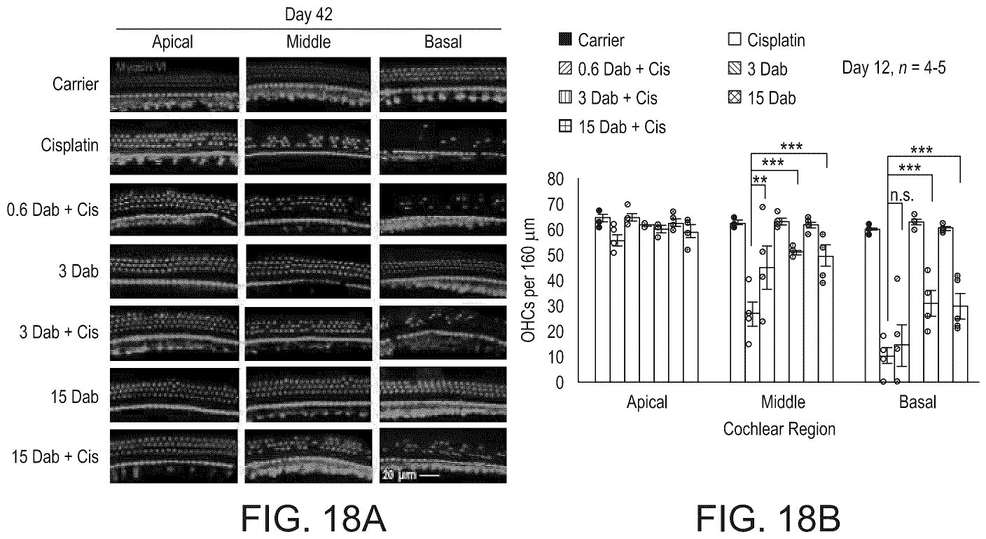

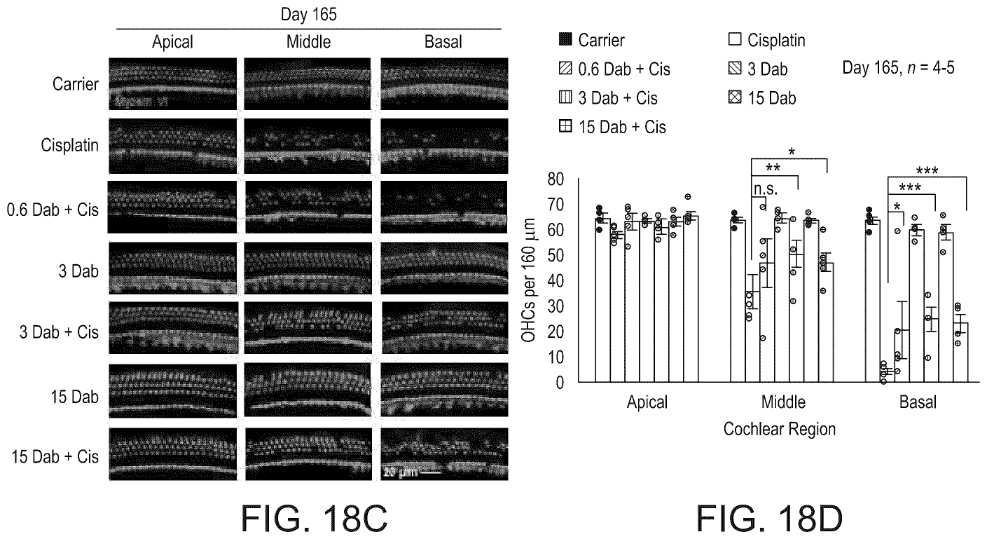

Now referring to FIGS. 17A-17C The last functional test was EP to determine whether cisplatin caused functional damage to the stria vascularis after the multicycle cisplatin protocol. FIG. 18A shows an example EP recording depicting the microelectrode insertion and withdrawal from the scala media through the basilar membrane (organ of Corti). Before any treatment began, EP from 6 mice were recorded with an average potential of 103 mV with no difference between males and females (FIG. 17B). EP was recorded again in carrier and cisplatin alone treated mice immediately and 4 months after the completion of cycle 3. There was no change in EP for mice treated with cisplatin at all-time points tested (FIG. 17C).

Now referring to FIGS. 18A-18D is it show that Dabrafenib protects against cisplatin-induced outer hair cell loss. After all functional tests were performed, cochleae were dissected for analysis of OHCs. At day 42, mice co-treated with 15 and 3 mg/kg dabrafenib and cisplatin had significantly more OHCs at the basal region compared to cisplatin alone treated mice, while 15, 3, and 0.6 mg/kg dabrafenib also had significantly more OHCs at the middle region (FIGS. 18A and 18B).

Cisplatin alone treated mice had an average of 36±7 and 4±1 OHCs following cisplatin treatment at the 16 and 32 kHz regions per 160 μm, respectively. At the 16 and 32 kHz regions, mice treated with 15 mg/kg dabrafenib had 47±4 and 23±4 OHCs following treatment, while mice treated with 3 mg/kg dabrafenib had 51±6 and 25±5 OHCs per 160 μm, respectively. 0.6 mg/kg dabrafenib treated mice had slightly less OHCs compared to the higher dabrafenib doses, 47±10 at 16 kHz and 21±11 at 32 kHz. At day 165, 15 and 3 mg/kg treated mice had significantly more OHCs at the basal and middle region of the cochlea compared to the cisplatin alone treated mice. 0.6 mg/kg dabrafenib conferred protection from OHC loss at the middle region but not at the basal region (FIGS. 18C & 18D). At the 16 and 32 kHz regions, cisplatin alone treated mice have 27±5 and 10±3 OHCs per 160 μm, while mice treated with 15 mg/kg dabrafenib have 50±4 and 30±5 OHCs, respectively. The mice treated with 3 mg/kg dabrafenib have 51±1 OHCs at the 16 kHz region and 31±5 at the 32 kHz region, while 0.6 mg/kg dabrafenib treated mice have 45±8 OHCs at 16 kHz and 15±8 at 32 kHz.

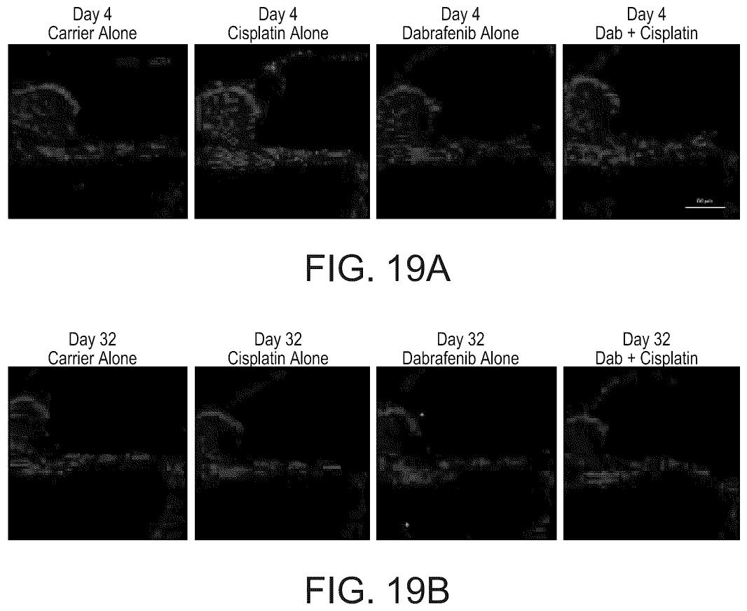

Now referring to FIGS. 19A-19B dabrafenib mitigates cisplatin-induced phosphorylation of ERK. Cochleae were collected from mice at the end of treatment cycles 1 and 3, day 4 and 32 respectively, to examine cisplatin and dabrafenib's effect on phosphorylation of the downstream target ERK. On day 4, cisplatin treated mice had increased ERK phosphorylation in the organ of Corti region of the middle turn compared to other cohorts. Co-treatment of 3 mg/kg dabrafenib with cisplatin mitigated phosphorylation of ERK in the organ of Corti; similarly, elevated phosphorylation of ERK was not observed in carrier and 3 mg/kg dabrafenib treated mice (FIG. 19A). Changes in ERK phosphorylation were not observed in other regions of the cochleae, including the stria vascularis, spiral limbus, spiral ligament, and spiral ganglion neurons on day 4. Increased ERK phosphorylation was not observed in any cohort on day 32, including cisplatin treated mice (FIG. 19B). Together, the data demonstrates cisplatin induces phosphorylation of ERK in the organ of Corti early in cycle 1 and that dabrafenib co-treatment mitigates this change in MAPK signaling.

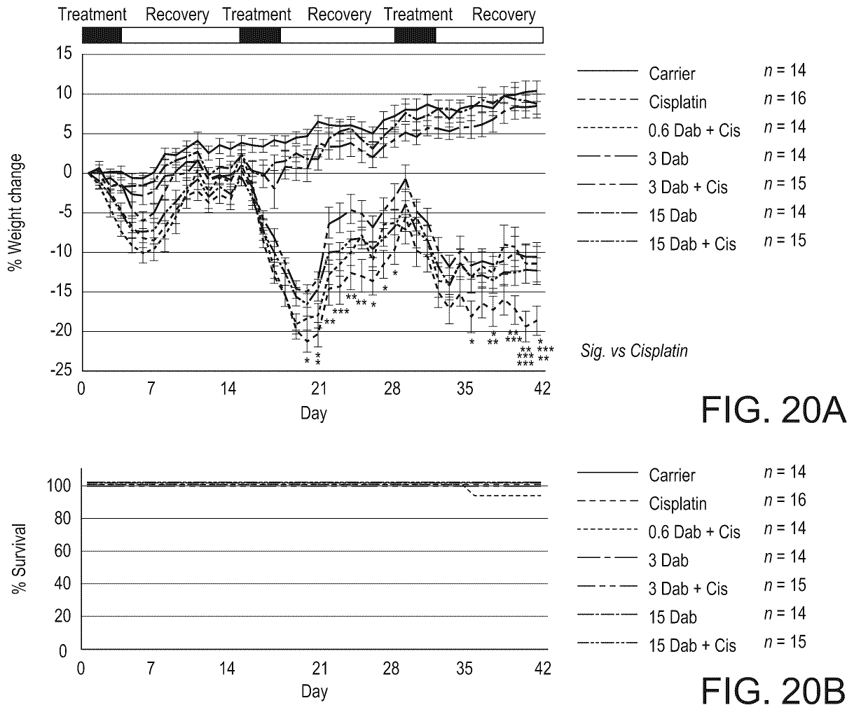

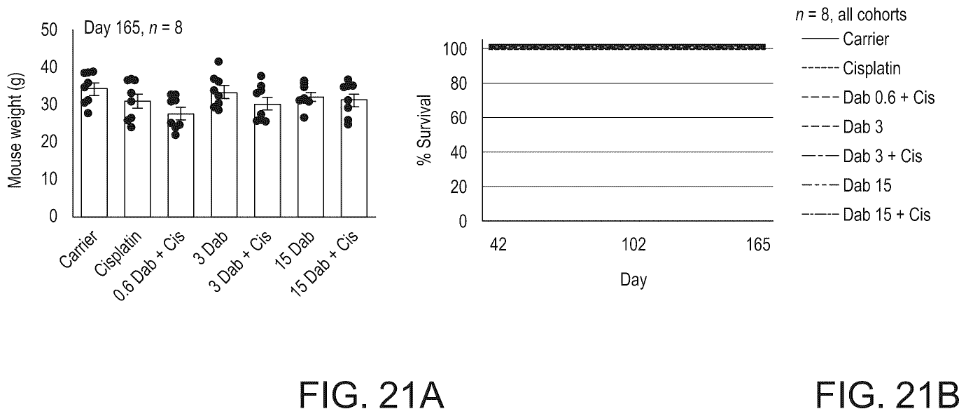

Now referring to FIGS. 20A-20B and FIGS. 21A and 21B dabrafenib does not increase systemic toxicity when combined with cisplatin. Throughout the multi-cycle treatment protocol, mice are weighed daily to analyze weight loss for each cohort. Cisplatin treated mice lost up to 21% body weight throughout the treatment regimen. Carrier and dabrafenib (3 and 15 mg/kg) alone treated mice did not exhibit weight loss, but rather steadily gained weight. All 3 doses of dabrafenib (15, 3, and 0.6 mg/kg) showed significantly less weight loss on multiple days in mice co-treated with cisplatin compared to cisplatin alone (FIG. 20A). Dabrafenib at 3 mg/kg demonstrated the best protection from weight loss with co-treated mice losing only 15% of their original body weight throughout both cycles 2 and 3 (FIG. 20A). Mice were again weighed on day 165 and all cohorts exhibited similar weights with no significant difference between groups (FIG. 21A). There was no significant mouse death in any treatment group throughout the protocol.

Now referring to FIGS. 22A and 22B and FIGS. 23A and 23B, mouse liver and kidneys were collected to analyze the toxic effect of cisplatin and dabrafenib on these organs. Kidneys were stained with hematoxylin and eosin (H&E) and Periodic acid-Schiff (PAS) with FIG. 22A showing representative images for each group immediately after cycle 3. Samples were then analyzed by a trained and experienced pathologist, who was blinded to the experimental conditions to determine the amount of damage in each group. There was no significant kidney damage in any cohort at both day 42 and 165 Livers were stained with H&E and Masson's Trichrome stain with FIG. 21D showing representative images at day 42. There was no significant difference in the amount of liver damage between all experimental groups as indicated by the histology score at both day 42 and day 165 (FIG. 21E and FIG. 21F).

Now referring to FIG. 24A Representative images of livers dissected and stained at day 165 of the protocol. Representative H&E stained images of the liver when dissected and stained at day 165 (4-months post cisplatin treatment). The 6 treatment groups analyzed are the following: carrier alone, cisplatin alone, 15 mg/kg dabrafenib alone, 3 mg/kg dabrafenib alone, 15 mg/kg dabrafenib and cisplatin co-treatment, and 3 mg/kg dabrafenib and cisplatin co-treatment. FIG. 24B shows representative Masson's Trichrome stained images of the liver at day 165 of experimental protocol.

Experimental summary: To conclude, dabrafenib is a therapeutic candidate for preventing cisplatin-induced hearing loss. It has a low effective dose of one tenth of the human equivalent dose (3 mg/kg administered twice day), a good toxicity profile, a therapeutic index of at least 25 in the multi-dose cisplatin regimen, protects both female and male mice, reduces hearing loss in two different strains of mice (FVB/NJ and CBA/CaJ), offers protection from weight loss that occurs during cisplatin chemotherapy, and persistence of hearing protection for at least four months after cisplatin treatments.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method to prevent cisplatin-induced hearing loss comprising: orally administering to subject in need thereof a pharmaceutical composition comprised of: a therapeutically effective amount of dabrafenib to prevent hearing loss due to cisplatin treatment,
wherein the therapeutically effective amount of dabrafenib is administered to the subject in a first cycle.

2. The method of claim 1, wherein the therapeutically effective amount of dabrafenib administered to the subject in the first cycle is in a range from about 0.6 mg/kg to about 60 mg/kg in mice or a human equivalent dose.

3. The method of claim 2, wherein the therapeutically effective amount of dabrafenib in the first cycle is about 12 mg/kg in mice or a human equivalent dose.

4. The method of claim 1, wherein the treatment amount of cisplatin is about 30 mg/kg in mice or a human equivalent dose.

5. The method of claim 1, wherein dabrafenib is administered forty-five minutes prior to the cisplatin treatment.

6. The method of claim 1, wherein dabrafenib is co-administered with the cisplatin treatment.

7. The method of claim 1, further comprising the step of orally administering to subject in need thereof a pharmaceutical composition comprised of: a sufficient amount of dabrafenib to prevent hearing loss due to cisplatin treatment, wherein the sufficient amount of dabrafenib is about 12 mg/kg in mice or a human equivalent dose for a second cycle after a rest period.

8. The method of claim 5, further comprising the step of orally administering to subject in need thereof a pharmaceutical composition comprised of: a sufficient amount of dabrafenib to prevent hearing loss due to cisplatin treatment, wherein the sufficient amount of dabrafenib is about 12 mg/kg in mice or a human equivalent dose for a third cycle after a rest period.

9. A method to prevent cisplatin-induced hearing loss comprising: orally administering to subject in need thereof a pharmaceutical composition comprised of: a sufficient amount of Dabrafenib to prevent hearing loss due to cisplatin treatment, wherein the sufficient amount of dabrafenib is in a range from 0.6 mg/kg to 60 mg/kg in mice or a human equivalent dose.

10. The method of claim 9, wherein the treatment amount of cisplatin is about 30 mg/kg in mice or a human equivalent dose.

11. The method of claim 9, wherein dabrafenib is administered forty-five minutes prior to the cisplatin treatment.

12. The method of claim 9, wherein dabrafenib is co-administered with the cisplatin treatment.

13. The method of claim 9, further comprising the step of orally administering to subject in need thereof a pharmaceutical composition comprised of: a sufficient amount of dabrafenib to prevent hearing loss due to cisplatin treatment, wherein the sufficient amount of dabrafenib is about 3 mg/kg in mice or a human equivalent dose for a second cycle after a rest period.

14. The method of claim 13, further comprising the step of orally administering to subject in need thereof a pharmaceutical composition comprised of: a sufficient amount of dabrafenib to prevent hearing loss due to cisplatin treatment, wherein the sufficient amount of dabrafenib is about 3 mg/kg in mice or a human equivalent dose for a third cycle after a rest period.

15. The method of claim 1 comprising administering to the subject a therapeutically effective amount of AZD5438.

16. The method of claim 15, wherein the therapeutically effective amount of AZD5438 ranges from about 5 mg/kg to about 85 mg/kg, preferably about 35 mg/kg.

17. The method of claim 9 comprising administering to the subject a therapeutically effective amount of AZD5438.

18. The method of claim 17, wherein the therapeutically effective amount of AZD5438 ranges from about 5 mg/kg to about 85 mg/kg, preferably about 35 mg/kg

19. A method to prevent cisplatin-induced hearing loss comprising: orally administering to subject in need thereof a pharmaceutical composition comprised of: a sufficient amount of dabrafenib to prevent hearing loss due to cisplatin treatment, wherein the sufficient amount of Dabrafenib is about 0.6 mg/kg in mice or a human equivalent dose.

20. The method of claim 19, wherein the treatment amount of cisplatin is about 30 mg/kg in mice or a human equivalent dose.

21. The method of claim 19, wherein dabrafenib is administered forty-five minutes prior to the cisplatin treatment.

22. The method of claim 19, wherein dabrafenib is administered simultaneously with the cisplatin treatment.

23. The method of claim 19, further comprising the step of orally administering to subject in need thereof a pharmaceutical composition comprised of: a sufficient amount of dabrafenib to prevent hearing loss due to cisplatin treatment, wherein the sufficient amount of dabrafenib is about 0.6 mg/kg in mice or a human equivalent dose for a second cycle after a rest period.

24. The method of claim 19, further comprising the step of orally administering to subject in need thereof a pharmaceutical composition comprised of: a sufficient amount of dabrafenib to prevent hearing loss due to cisplatin treatment, wherein the sufficient amount of dabrafenib is about 0.6 mg/kg in mice or a human equivalent dose for a third cycle after a rest period.

25. A method of maintaining a subject's body weight during cisplatin treatment comprising: co-administrating a sufficient amount of cisplatin for the desired treatment and a sufficient amount of a B-raf inhibitor to prevent weight loss during cisplatin treatment.

26. A pharmaceutical composition in a unit dose form comprising cisplatin and an effective amount of a B-raf inhibitor to treat hearing loss caused by the co-administration of cisplatin to a subject in need of treatment thereof wherein the B-raf inhibitor is selected from the group consisting of: Dabrafenib, Vemurafenib, PLX-4720 and RAF-265.

27. The pharmaceutical composition of claim 26 further comprising: a sufficient amount of Trametinib to treat hearing loss due to cisplatin treatment.

28. The pharmaceutical composition of claim 27 wherein the sufficient amount of Trametinib is about 2 mg/kg in mice or an equivalent dosage for a human twice daily.

29. The pharmaceutical composition of claim 26, further comprising a therapeutically effective amount of AZD5438.

30. A method of treating a side effect from a cisplatin-induced chemotherapy treatment in a subject in need thereof, comprising administering to the subject a sufficient amount of cisplatin to treat cancer in the subject and one or a plurality of treatment cycles comprising administering a B-raf inhibitor to the subject.

31. The method of claim 30, wherein the B-raf inhibitor is dabrafenib.

32. The method of claim 20, further comprising administering to the subject a compound selected from trametinib or AZD5438.
